(19) United States
(12) Patent Application Publication
(10) Pub. No.: US 2008/0081958 A1
(43) Pub. Date: Apr. 3, 2008

(54) Implantable medical device with sensor self-test feature

(75) Inventors: Timothy J. Denison, Minneapolis, MN (US); Brian B. Lee, Golden Valley, MN (US); Keith A. Miesel, St. Paul, MN (US); Eric J. Panken, Edina, MN (US)

Correspondence Address: SHUMAKER & SIEFFERT, P. A., 1625 RADIO DRIVE, SUITE 300, WOODBURY, MN, 55125, US

(73) Assignee: Medtronic, Inc., Minneapolis, MN (US)

(21) Appl. No.: 11/789,438

(22) Filed: Apr. 24, 2007

Related U.S. Application Data
(60) Provisional application No. 60/847,817, filed on Sep. 28, 2006.

(51) Int. Cl. A61B 5/00

(52) U.S. Cl. 600/300

(57) ABSTRACT

An implantable medical device (IMD) applies a sensor self-test when a sensing device generates a sensor signal indicating an event, or when the sensor is used to validate an event detected by another device. The event may be based on a sensed condition that triggers an operational adjustment, such as a therapy or diagnostic adjustment within the IMD. A sensor self-test verifies that an implantable sensing device is functional, and can be performed with or without activating the sensor. Activating the sensor may involve, application of an electrical input signal that causes the sensor to generate an output signal. Alternatively, the sensor self-test may be performed without activating the sensor by analyzing the continuity of a signal path between the sensor and sensor interface circuitry. In either case, a sensor self-test verifies proper operation so that operational adjustments can be made with greater confidence.

This application claims the benefit of U.S. Provisional Application No. 60/847,817, filed Sep. 28, 2006, the entire content of which is incorporated herein in its entirety.

### TECHNICAL FIELD

The invention relates to implantable medical devices (IMDs) and, more particularly, implantable medical devices including sensors.

### BACKGROUND

IMDs such as electrical stimulation devices, drug delivery devices and diagnostic monitoring devices often include implanted sensors to sense a variety of physiological conditions. Examples of implantable sensors include accelerometers, pressure sensors, flow sensors, heart sound sensors, sense electrodes, electrochemical sensors, biological agent sensors, and the like. As an example, an accelerometer may be used in conjunction with a cardiac stimulation device or neurostimulation device to indicate the activity level or posture of a patient. As another example, a glucose sensor may be used in conjunction with an insulin delivery device to indicate a glucose level.

In response to sensor information, an IMD may deliver, terminate or adjust therapy deliver to the patient, activate or modify diagnostic recording, or activate a notification or alarm. For example, an insulin delivery device may adjust dosage or rate in response to an indicated glucose level. As another example, a cardiac stimulation system may adjust cardiac pacing on a rate-responsive basis in response to an indicated activity level. Similarly, a neurostimulation device may adjust stimulation parameters in response to indication of a posture or activity change. Hence, performance of an implanted sensor can impact diagnostic or therapeutic efficacy of an implanted medical device.

### SUMMARY

This disclosure describes a sensor self-test feature for use with an IMD. The IMD may include or be coupled to a sensing device that generates a sensor signal indicative of a physiological condition. The IMD may apply a sensor self-test when the sensing device generates a sensor signal indicating an event, or when the sensor is used to validate an event detected by a different sensing device. The event may be based on a sensed condition that triggers therapy initiation, therapy termination or control of one or more therapy parameters, each of which may be considered a therapy adjustment.

A sensor self-test verifies that an implantable sensing device is functional, and can be performed with or without activating the sensor. Activating the sensor may involve, for example, application of an electrical input signal that causes the sensor to generate an output signal. Alternatively, the sensor self-test may be performed without activating the sensor by analyzing the continuity of a signal path between the sensor and sensor interface circuitry. In either case, a sensor self-test verifies proper operation so that therapy adjustments based on events indicated by a sensing device can be made with greater confidence.

In one embodiment, the invention provides an implantable medical device comprising a sensing device that generates a sensor signal indicative of a physiological condition, and a therapy delivery device that detects an event associated with an operational adjustment of an implantable medical device, wherein the sensing device performs a self-test of the sensing device in response to the event, and the therapy delivery device determines whether to make the operational adjustment based on the sensor signal and a result of the self-test.

In another embodiment, the invention provides a method comprising detecting an event associated with an operational adjustment of an implantable medical device, obtaining a sensor signal indicative of a physiological condition from a sensing device, performing a self-test of the sensing device in response to the event, and determining whether to make the operational adjustment based on the sensor signal and a result of the self-test.

In an additional embodiment, the invention provides a method comprising obtaining a sensor signal indicative of a physiological condition from a sensing device, and performs a self-test of the sensing device in response to an event associated with an operational adjustment of an implantable medical device.

In another embodiment, the invention provides an implantable medical device comprising a sensing device that generates a sensor signal indicative of a physiological condition, and self-test circuitry that performs a self-test of the sensing device in response to an event associated with an operational adjustment of the implantable medical device.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating an IMD including a sensing device.

FIG. 2 is a block diagram illustrating an example sensing device incorporating an accelerometer.

FIG. 3 is a block diagram illustrating an accelerometer channel for use in the sensing device of FIG. 2.

FIG. 4 is a block diagram illustrating an IMD including a sensing device with a self-test feature.

FIG. 5 is a block diagram illustrating a self-test feature that applies a test signal to activate a sensor in a sensing device.

FIG. 6 is a block diagram illustrating a self-test feature that applies a test signal to interface circuitry to verify interface integrity without activating a sensor in a sensing device.

FIG. 7 is a flow diagram illustrating a technique for performing a sensor self-test procedure via sensor activation.

FIG. 8 is a flow diagram illustrating a technique for performing a sensor self-test procedure without sensor activation. A

FIG. 9 is a flow diagram illustrating a technique for using a self-test sensing device to trigger or validate a medical event.

FIG. 10 is a flow diagram illustrating an technique for using a self-test sensing device to trigger or validate a cardiac arrhythmia as a medical event.

FIG. 11 is a flow diagram illustrating an exemplary technique for using a self-test sensing device to trigger or validate a posture change as a medical event.

### DETAILED DESCRIPTION

This disclosure describes a sensor self-test feature for use with an IMD. The IMD may include or be coupled to a sensing device that generates a sensor signal indicative of a physiological condition. The IMD may apply a sensor self-test when the sensing device generates a sensor signal indicating an event, or when the sensor is used to validate an event detected by a different sensing device. The event may be based on a sensed condition that triggers therapy initiation, therapy termination or control of one or more therapy parameters, each of which may be considered a therapy adjustment.

A sensor self-test verifies that an implantable sensing device is functional, and can be performed with or without activating the sensor. Activating the sensor may involve, for example, application of an electrical input signal that causes the sensor to generate an output signal. Alternatively, the sensor self-test may be performed without activating the sensor by analyzing the continuity of a signal path between the sensor and sensor interface circuitry. In either case, a sensor self-test verifies proper operation so that therapy adjustments based on events indicated by a sensing device can be made with greater confidence.

The sensing device may include a sensor that generates an electrical signal indicative of a physiological condition and sensor circuitry that performs a self-test to verify that the device is operational. Examples of implantable sensors include accelerometers, pressure sensors, blood flow sensors, heart sound sensors, electrocardiogram (ECG) sense electrodes, electroencephalogram (EEG) sense electrodes, electrochemical sensors, biological agent sensors, and the like. Accelerometers will be generally described in this disclosure for purposes of illustration, but without limitation of the invention as broadly embodied and described in this disclosure.

A sensor may be used to detect conditions useful in adjusting therapy or diagnosis for different diseases or disorders, such as cardiac arrhythmia, cardiac fibrillation, chronic pain, tremor, Parkinson's disease, epilepsy, urinary or fecal incontinence, sexual dysfunction, obesity, gastroparesis, or diabetes. Example physiological conditions include posture or movement of a patient, heart sounds, blood pressure, brain potentials, nerve potentials, chemical levels, or other physiological conditions. A sensing device may be implemented with a single sensor or a combination of sensors, such as accelerometers, piezoelectric sensors, strain gauge sensors, sense electrodes, electrochemical sensors, biological sensors, and other sensors.

A self-test feature may be used for sensors associated with a variety of IMDs. A neurostimulator, for example, may be configured to deliver a variety of therapies such as spinal cord stimulation, deep brain stimulation, pelvic floor stimulation, gastric stimulation, peripheral nerve stimulation, or other forms of neurostimulation. A cardiac stimulator may be configured to deliver cardiac pacing, cardioversion/fibrillation, antitachycardia packing, cardiac resynchronization, or other forms of cardiac stimulation. As further alternatives, an IMD may be a drug delivery device, a muscle or organ stimulator, a diagnostic loop recorder, or another type of IMD.

Therapy delivered by an IMD may be adjusted in response to an event indicated by a sensor in a sensing device. The event may a physiological condition or level that indicates a need for adjustment. Therapy adjustment may include initiation of therapy, termination of therapy, or control of one or more parameters associated with therapy, such as amplitude, pulse rate, pulse width, electrode combination, duration, dosage, or the like. As an example, a neurostimulator may adjust amplitude, pulse rate, and/or pulse width, or select a different neurostimulation program, when an accelerometer indicates a change in activity level or posture, e.g., from sitting to standing. In some cases, in response to change in activity level or posture, a neurostimulator may select a different neurostimulation program, defining a different set of parameters, or initiate or terminate neurostimulation.

For a cardiac stimulator, activity level or posture may be used to trigger adjustment of cardiac pacing therapies. For example, a cardiac pacemaker may apply rate-responsive pacing to increase or decrease pacing rate according to a patient's activity level, which may be sensed by an accelerometer. Also, a cardioverter/defibrillator may use a sudden posture change indicating a fall to verify whether a defibrillation shock should be delivered. Similar events also may be useful in triggering a loop recorder to record diagnostic information such as sensor output signals, or to increase recording frequency or sensitivity. Adjustment of therapy or diagnostic operation may be generally referred to as an operational adjustment.

Different types of sensors may drive other therapy or diagnostic adjustments. For a drug delivery device, a sensing device may indicate an activity level, posture or concentration of a substance, in which case dosage or rate of fluid delivery may be adjusted. For example, a glucose sensor may be used in conjunction with an insulin delivery device to trigger increased or decreased insulin dosage or rate in response to a sensed glucose level. Accordingly, a self-test sensor may be useful in a wide variety of settings in which adjustments to therapy and/or diagnostic operations may be made based on sensor output. In each case, the self-test may verify proper sensor operation and prevent therapy or diagnostic adjustments in reliance on erroneous output from a malfunctioning sensor.

A sensing device, in accordance with this disclosure, may be configured for implantation within a patient, either within an IMD or in conjunction with an IMD. For example, the sensing device may be contained in a housing of an IMD, positioned on a housing of an IMD, or deployed in a lead or catheter extending from an IMD housing. As further alternative, the sensing device may be a separately implantable device that communicates with an IMD and/or an external controller via wireless telemetry. In each case, the sensing device may be configured to perform a self-test to verify that the sensing device, including one or more sensors within the sensing device, is operable. The self-test may be performed periodically. Alternatively, or additionally, the self-test may be performed in the course of adjusting therapy or diagnostic operations in response to sensor output. As a further alternative, the sensing device may perform a self-test in response to a command received from an external controller. The command may be generated in response to a detected event, which may be indicated by the output of the self-test sensing device or the output of another sensing device.

The sensing device and sensor circuitry may perform the self-test by activating the sensor or not activating the sensor. The sensor circuitry may activate the sensor by applying an electrical input signal to the sensor that activates the sensor such that the sensor produces a sensor signal. Using a micro-electro-mechanical system (MEMS) accelerometer as an example, the sensor circuitry may apply an electrostatic voltage to one or more fixed fingers that inter-digitate with a plurality of beam fingers attached to a proof mass. The electrostatic voltage causes the proof mass to deflect, thereby changing the capacitance measured between the beam fingers and the fixed fingers. Because the electrostatic voltage applied to the sensor is known a priori, the change in capacitance is also known. Thus, the sensing device can determine the operational state of the accelerometer by comparing the measured change in capacitance to a pre-determined threshold value or range of values.

Alternatively, the sensor circuitry may perform a self-test without activating the sensor by analyzing the continuity of a signal path from the sensor. The signal path is the path a sensor signal follows from the sensor to sensor circuitry that processes the signal. In general, a sensor signal is processed by the sensor circuitry to provide an output signal that is suitable for use by the IMD. For example, the sensor circuitry may amplify, filter, and otherwise process the sensor signal to produce a stable, low noise sensor output signal that can be used by the IMD. If a signal applied to the signal path of the sensor successfully propagates to the sensor circuitry, electrical continuity of the signal path is confirmed. In this manner, sensor operation can be confirmed, at least in part, without activating the sensor.

The self-test may be performed to verify proper sensor operation before proceeding with an operational adjustment, such as a therapy or diagnostic adjustment, based on the sensor output. For example, the IMD may detect an event based at least in part on output from the sensing device or output from a different device, such as a different sensing device. The IMD may make a therapy or diagnostic adjustment in response to the event.

If the event is a posture change indicated by a sensing device, for example, the IMD may proceed to adjust neurostimulation therapy by adjusting one or more therapy parameters, initiating therapy, terminating therapy, or changing to a different therapy program, which may specify different parameters. Before adjusting therapy, however, it may be desirable to first confirm proper operation of the sensing device by applying the sensor self-test feature. If the sensor is found to be operable, the IMD proceeds with the adjustment. If the sensor is not operable, however, the IMD may be configured to avoid the adjustment.

If the posture change or a different event was indicated by a different device, it also may be desirable to use the self-test sensing device to validate the event triggered by the other device. In this case, the sensor self-test feature may be applied to first determine whether the sensing device is operable. If so, the IMD may then correlate the sensing device output with the event triggered by the output of the other device to determine whether the event is valid. Hence, the sensing device self-test feature may be used when the sensing device triggers, i.e., indicates, an event, such as a posture change, or when the sensing device is used to validate an event triggered by another sensing device. The sensor signal generated by the sensing device may indicate the event, or a sensor signal generated by the sensing device may validate the event.

As another example, if the medical event is cardiac fibrillation sensed by sense electrodes, the IMD may proceed to deliver a cardiac defibrillation shock to the patient. Before delivering a painful defibrillation shock, however, IMD may seek to validate the defibrillation event by interrogating other sensing devices, such as an accelerometer for indication of a sensed posture change or fall consistent with fibrillation, or a heart sound or respiration monitor for indication of sensed heart sounds or respiratory changes consistent with fibrillation. As an illustration, if a patient is experiencing fibrillation, it may be generally expected that an accelerometer should generate a signal indicative of inactivity, a slumped or reclined or prone posture, and possibly a sudden fall.

In this case, another sensing device such as an accelerometer may be used to validate the fibrillation indicated by the sense electrodes in order to avoid an unnecessary shock. However, it may be desirable to apply the sensor self-test feature to the accelerometer in conjunction with the validation to ensure proper sensor operation. If the sensing device is working properly and generates output consistent with the triggered medical event, the IMD may proceed with delivery of a defibrillation shock. If the sensing device is working properly and does not generate output consistent with the triggered medical event, the IMD may withhold a defibrillation shock pending further analysis. If the sensing device is not working properly, the IMD may proceed with delivery of a defibrillation shock.

A sensor self-test feature may be desirable not only in a therapeutic or diagnostic application, as described above, but also during a manufacturing process to identify potentially defective sensing devices. In particular, a self-test that does not require activation of the sensor may be useful. In this case, a signal can be applied to the sensor and monitored for propagation to the sensor output for purposes of verifying continuity of the sensor signal path. A self-test feature that does not require sensor activation may streamline the manufacturing process by reducing the number of physical tests needed to verify that one or more sensing devices are functional.

Physical tests, such as shake/tilt tests for an accelerometer, are both time consuming and costly. Thus, reducing the number of physical tests can result in improved efficiency and reduced cost. Although the non-activating self-test may not entirely replace physical tests, the non-activating self-test may replace at least some of the physical tests performed during a manufacturing process. For example, a manufacturer may produce a batch or large quantity of sensing devices and physically test a number of the sensing devices. The number of devices physically tested may be large. To increase efficiency and reduce cost, the non-activating self-test may replace at least some of the physical tests. In this way, the manufacturer may physically test a reduced sample of the sensing devices and use the non-activating self-test to test other sensing devices.

In addition to self-test features, a sensing device may also include a reset feature. The reset feature may be incorporated as a step in an operational adjustment algorithm, e.g., for therapy or diagnostic adjustment, to reset the sensing device to an operational state. A sensing device, such as a MEMS accelerometer, may become inoperable when inter-digitated fingers become stuck to each other due to stiction caused by Van der Waal forces. Stiction may result from a sudden motion, such as the device being jolted or dropped, or from severe shock and electrical overstress. In this case, the reset feature may involve powering the sensor off, thereby releasing the inter-digitated fingers from each other, and powering the sensor back on to return the sensor to an operational state.

In a therapy or diagnostic adjustment algorithm, the sensing device may execute the reset feature after performing a self-test that indicates the device is inoperable. As an example, sensor circuitry may be configured to toggle a sensor reference or excitation voltage on and off. If the reset fails, the reset may be attempted one or more times. However, if the device is not returned to an operational state after a number of reset attempts, the sensing device or associated IMD may generate an alert that indicates the device has failed. In the event that the reset is successful in returning the device to an operational state, the device may continue to operate normally and the therapy or diagnostic adjustment may be completed.

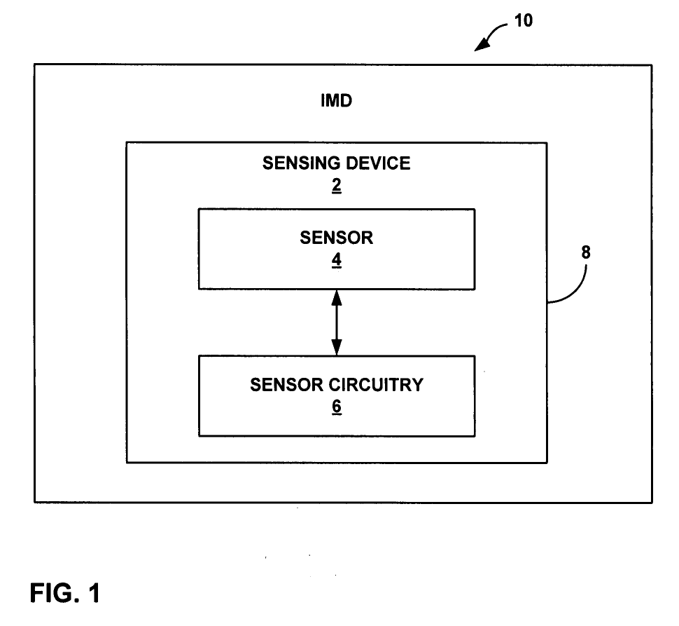

FIG. 1 is a block diagram illustrating a sensing device 2 with self-test features. In general, sensing device 2 is implanted within a patient and performs a self-test, also referred to as a self-diagnostic test or an integrity test, to verify that sensing device 2 is functional. In the example of FIG. 1, sensing device 2 includes a sensor 4 and sensor circuit 6 mounted on a substrate 8 within an IMD 10. Alternatively, sensing device 2 may be mounted on IMD 10, deployed within a lead or catheter coupled to IMD 10, or separately implanted but equipped for communication with IMD 10 via wireless telemetry. The self-test can be performed periodically, on a schedule, on command, as a verification step in a therapy delivery or diagnostic algorithm utilized by IMD 10, or to streamline the manufacturing of device 2.

Sensing device 2 may include sensor 4 and sensor circuitry 6. In general, sensor 4 generates an electrical signal indicative of a physiological condition within a patient. Sensor circuitry 6 processes the signal to generate a sensor output signal. In addition, sensor circuit 6 is configured to perform a self-test to verify that sensor 4 is functional. Sensor 4 may be realized by any of a variety of sensors, such an accelerometer, a piezoelectric sensor, a strain gauge, sense electrodes, electrochemical sensors, biological sensors, and other sensors capable of sensing a physiological parameter. IMD 10 may operate at least partially based on the output of sensing device 2, and may be a therapeutic IMD, a diagnostic IMD, or a combined therapeutic/diagnostic IMD. Accordingly, IMD 10 may deliver therapy to the patient and/or record data based on the output of sensing device 2.

IMD 8 may deliver therapy in the form of electrical pulses, shocks or waveforms, drugs, or a combination of both to a variety of tissue sites, such as the heart, the brain, the spinal cord, pelvic nerves, peripheral nerves, or the gastrointestinal tract of the patient. IMD 10 may be configured as a drug delivery device, cardiac pacemaker, cardioverter/defibrillator, spinal cord stimulator, pelvic nerve stimulator, deep brain stimulator, gastrointestinal stimulator, peripheral nerve stimulator, or muscle stimulator. IMD 10 may deliver therapy to support different therapeutic applications, such as cardiac stimulation, deep brain stimulation (DBS), spinal cord stimulation (SCS), pelvic stimulation for pelvic pain, incontinence, or sexual dysfunction, gastric stimulation for gastroparesis, obesity or other disorders, or peripheral nerve stimulation for pain management. Stimulation may also be used for muscle stimulation, e.g., functional electrical stimulation (FES) to promote muscle movement or prevent atrophy.

Sensing device 2 may generally be configured for implantation within a patient. Thus, sensing device 2 may be formed in a package that can be contained in or on a housing of IMD 10, or in a lead or catheter (not shown) extending from the housing of IMD 10. In some embodiments, sensing device 2 may be disposed on or within a distal lead tip of a lead (not shown) extending from IMD 10. The size of sensing device 2 may vary. For example, the size may be dependent on factors such as sensor type, and the complexity of sensor circuitry 6. As an example, the size of sensing device 2 may be different if sensor 4 is implemented as a two-axis accelerometer or a three-axis accelerometer. In another example, the size of sensing device 2 may be different when sensor 4 is implemented as a pressure sensor instead of an accelerometer. However, sensing device 2 is preferably miniaturized for ease of implantation within a patient. In an exemplary embodiment, sensing device 2 may be realized by an accelerometer formed on a substrate molded into a package that is less than or equal to approximately 3 millimeters (mm) in length, less than or equal to approximately 3 mm in width, and less than or equal to approximately 1 mm in height.

Sensor 4 and sensor circuitry 6 may be coupled to each other and packaged on common substrate 8. In general, sensor 4 converts mechanical energy into an analog output voltage that is processed by sensor circuitry 6 to generate a sensor output signal. As previously described, IMD 10 may make an operational adjustment by adjusting therapy or diagnostic operations based on the sensor output signal. In an example embodiment, sensor 4 may be fabricated using micro-electro-mechanical systems (MEMS) technology. MEMS technology uses micromachining processes to selectively etch away parts of substrate 8, which may be a silicon substrate, or add new structural layers to form mechanical and electromechanical devices. MEMS technology integrates mechanical elements, sensors, actuators, and electronics on a common substrate, such as substrate 8.

As examples, sensor 4 may comprise a two-axis accelerometer, a three-axis accelerometer, a pressure sensor, a flow sensor, a sense electrode or electrodes, an electrochemical sensor, or any other sensor capable of measuring a physiological parameter by measuring mechanical, chemical, thermal, biological, and/or magnetic phenomena. As previously described, example physiological parameters include posture, movement or activity, heart sounds, blood pressure, chemical levels, heart signals, brain signals, nerve signals, chemical levels, and other physiological parameters of the patient in which device 2 is implanted. Sensor 4 will be described as an accelerometer for purposes of illustration.

Sensor circuitry 6 may be fabricated using integrated circuit process sequences and may be coupled to sensor 4 via industry standard wire bonds. Sensor circuitry 6 generally includes self-test circuitry for performing a self-test and low power interface circuitry suitable for medical applications. The self-test features are described in detail below. The interface circuitry in sensor circuitry 6 provides a signal path that processes the signal generated by sensor 4 to produce a signal suitable for use by IMD 10 and draws a low current supply from a power source. For example, the signal path may amplify and filter the sensor signal to produce a stable, low noise signal that can be used by IMD 10 to identify an event, such as an arrhythmia, fibrillation, or other cardiac event, or make a decision, such as deliver therapy, record the signal for later retrieval and analysis, or generate a visible, audible or readable notification.

Sensor 4 may be manufactured separately from sensor circuitry 6. Sensor 4 then can be packaged on substrate 8 with sensor circuitry 6. Hence, sensor 4 and sensor circuitry 6 may be formed as separate components on substrate 8. Sensor 4 may be a MEMS sensor, such as a single or multiple axis accelerometer, while sensor circuitry 6 may be designed to interface with the sensor and an IMD. Sensor circuitry 6 may be designed to convert the output of sensor 4 into a stable, precise analog output signal while operating at low powers. In some embodiments, sensor 4 may be a capacitive based sensor, such as a MEMS accelerometer, and sensor circuitry 6 may transduce small capacitive deflections into a stable, low noise signal by amplifying and filtering the sensor output. Although sensor 4 and sensor circuitry 6 are described in this disclosure as separate components, sensor 4 and sensor circuitry 6 may alternatively be integrated with each other. That is, sensor 4 and sensor circuitry 6 may be fabricated together as a single component. In this case, the size of the package containing sensor 4 and sensor circuitry 6 may be reduced.

Self-test circuitry within sensor circuitry 6 may perform a self-test of sensor 4 by activating the sensor or without activating the sensor. The self-test circuitry may activate sensor 4 by applying an electrical input signal to sensor 4. The electrical input signal may also be referred to as a test signal. For example, sensor 4 may be a MEMS accelerometer and the electrical input (test) signal may be an electrostatic voltage applied to one or more fixed fingers that are inter-digitated with a plurality of beam fingers attached to a proof mass. The electrostatic voltage causes the proof mass to deflect, thereby changing the capacitance measured between the beam fingers and the fixed fingers.

Sensor 4 transduces the change in capacitance to a voltage that is measured by sensor circuitry 6 and, more particularly, the interface circuitry. Because the input (test) signal is known a priori, the signal generated by sensor 4 in response to the input signal can be compared to a predetermined threshold value or range of values to determine the operational state of device 2. In other words, sensor circuitry 6 can determine if device 2 is functional or not functional by examining the output of sensor 4 in response to an input (test) signal applied to sensor 4. If device 2 is determined to not be functional, sensor 4 may have failed due to failure of sensing elements, e.g., due to stiction or other physical failure. Alternatively, sensor circuitry 6 may have failed due to decoupling of wire bonds or other interconnections between sensor circuitry 6 and sensor 4, or due to failure of passive or active circuit components of sensor circuitry 6.

Although sensor 4 is generally described in this disclosure as a single- or multi-axis accelerometer, sensor 4 may be implemented as various other types of sensors. For example, the test signal may be applied to pressure sensors by injecting a test signal that electrostatically actuates one or more members of a capacitive diaphragm structure. As another example, the test signal may be applied to electroencephalogram (EEG), electromyography (EMG), or electrocardiogram (ECG) sense electrodes by superimposing small voltages on the sense electrodes. In each example, the output of the sensor 4 in response to such signals may be evaluated to verify that sensing device 2 is functional.

As an alternative, the self-test circuitry of sensor circuitry 6 may perform the self-test without activating sensor 4 by analyzing the continuity of the signal path between sensor 4 and sensor circuitry 6. The signal path is the path followed by the output of sensor 4 through the interface circuitry to sensor circuitry 6. Thus, the self-test circuitry may apply a test signal directly to the signal path, without mechanically or otherwise activating sensor 4, and examine the signal at the output of the signal path. If the signal successfully propagates to the output, electrical continuity is verified and the circuit is determined to be functional. In this way, the self-test verifies that no wire bonds or other circuit connections or components have failed. The circuit is determined to not be functional when the test signal or some processed, filtered, and/or amplified version of the signal does not successfully propagate to the output.

For self-test without activating sensor 4, the test signal generated by sensor circuitry 6 may be a reference voltage applied to one or more inputs of the interface circuitry or a signal with varying amplitude. In the case that the test signal is a reference voltage, the continuity of the signal path can be verified by comparing the voltage at the output to a predetermined voltage or range of voltages expected at the output of a properly functioning signal path. However, it is also contemplated that a more complex signal may be examined at the output to verify continuity of the signal path. As an example, instead of applying a constant reference voltage to the signal path, the test signal may be an electrical signal with varying amplitude, i.e., a varying waveform. In this case, the test signal may be a model of a waveform sensed by sensor 4 during normal operation, such as a cardiac waveform. Consequently, the test signal may be generated based on one or more example signals/waveforms, such as example cardiac waveforms. Again, because the test signal is known a priori, the output of the interface circuitry can be compared to an expected signal/waveform to determine the operational state of sensing device 2.

Sensing device 2 may perform a self-test periodically, on command, as a verification step in an operational adjustment algorithm utilized by IMD 10 for adjustment of therapy or diagnostic operation, or during the manufacturing process. For periodic testing, device 2 may perform self-tests according to a maintenance schedule or other schedule. The schedule may be stored in on-chip memory, i.e., memory within sensing device 2. Device 2 may also perform a self-test in response to a command, such as a command received from a programming device associated with sensing device 2 and/or IMD 10. The command may be generated automatically in response to a detected event. In this case, the self-test circuitry performs a self-test of the sensing device 2 in response to an event associated with an operational adjustment of IMD 2. Alternatively, the command may be generated automatically in response to input from a user, such as manual depression of one or more keys on an external programming device. Thus, the command may be initiated at any given time by a user.

When sensing device 2 performs a self-test as a validation step for a therapy or diagnostic adjustment in IMD 10, the self-test may be performed in response to detecting a sensed event that indicates a need for operational adjustment, e.g., either therapy or diagnostic adjustment. IMD 10 detects the event based at least in part on output received from sensing device 2, or on output received from a different device. In response to the triggering of the event by sensing device 2, IMD 10 directs sensor circuitry 6 to perform a self-test to verify the event triggered by the sensing device. If the event was triggered by a different device, IMD 10 directs sensor circuitry 6 to verify proper operation of sensing device 2 before validating an event triggered by the other device. Hence, sensor circuitry 6 may be responsive to a self-test command generated by IMD 10 upon detection of an event indicated by the output of sensing device 2 or indicated by the output of another device. Alternatively, sensor circuitry 6 may unilaterally initiate the self-test when sensing device 2 triggers, i.e., indicates, an event or when IMD 10 requests validation of a medical event by sensing device 2. In either case, IMD 10 can then render a decision to make an operational adjustment to therapy or diagnostic function with greater confidence if sensing device 2 is found to be operable.

If the sensor self-test indicates that sensing device 2 is not operable, IMD 10 or sensing device 2 may generate an alert that indicates that sensing device 2 has failed. The alert may be in the form of text displayed on a screen of an external programming device in response to a telemetry signal from sensing device 2, an audible alert, such as a beep or series of beeps, or other detectable alert, such as a vibration or vibration pattern, generated by an external programming device, sensing device 2 or IMD 10. Alternatively, or additionally, IMD 10 may record unfavorable results of the self-test feature for the attention of a medical care-giver upon interrogation of the IMD either remotely or upon a clinic visit by the patient.

Sensing device 2 also may be configured to perform a non-activated self-test during the manufacturing process. The non-activated self-test may streamline the manufacturing process by reducing the number of physical tests used to verify that one or more devices are functional. As an example, a sensing device 2 may be an accelerometer that is tested using a shake or tilt test. The shake/tilt test is both time consuming and costly. Therefore, when a batch or large number of sensing devices are manufactured, all the devices are not tested. Instead, the manufacturer may physically activate a sampling of the devices, while at least some of the devices may be tested using the non-activating self-test in place of a physical test. As mentioned previously, the non-activating self test may be performed by injecting a test signal at the input of interface circuitry associated with sensor circuitry 6 to evaluate the continuity of the signal path between sensor 4 and sensor circuitry 6.

In addition to the self-test features, sensing device 2 may also include a reset feature, as mentioned previously. The reset feature may return device 2 to an operational state. Reset may be particularly useful when senor 4 is a MEMS accelerometer that is inoperable because the inter-digitated fingers are stuck to each other. In this case, the reset feature may involve powering the sensor off to release the inter-digitated fingers from each other, and powering the sensor back on to return the sensor to an operational state. The reset feature may be used as part of the self-test procedure, and may involve toggling on and off a reference or excitation voltage applied to the accelerometer. The reset step may be executed a number of times in an attempt to return the device to an operational state. If the reset is successful in returning sensor 4 to an operational state, sensing device 2 may continue to operate normally. However, if the reset fails after repeated attempts, device 2 or IMD 10 may generate an alert as previously described, or record the malfunction within IMD 10 for later analysis.

Although sensing device 2 is shown in FIG. 1 as being contained in IMD 10, it should be understood that device 2 may be contained in a housing or “can” of IMD 10, on a housing of IMD 10, or on or within an implantable lead, catheter, or other therapy element extending from IMD 10. In each case, device 2 may have a wired connection to IMD 10. In another example, device 2 may be separately implanted and communicate by wireless telemetry with IMD 10 or another device that communicates with IMD 10. In this case, device 2 may transmit sensor signals to IMD 10 or an external controller and receive control signals from the IMD 10 or external controller to cause sensing device 2 to perform a self-test.

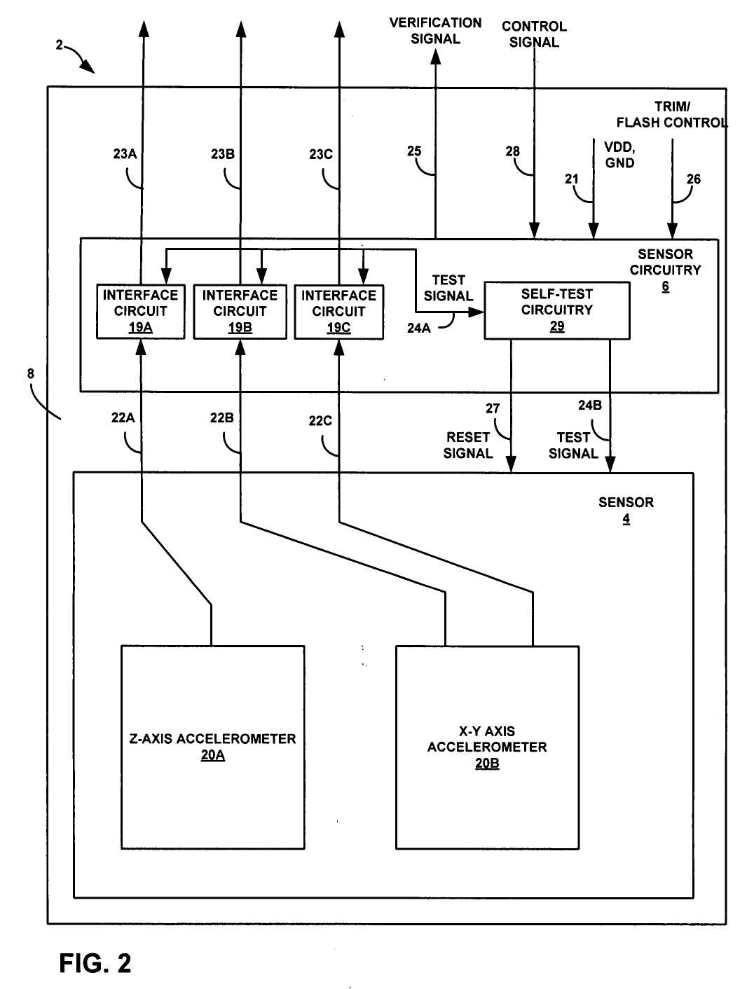

FIG. 2 is a block diagram illustrating an example sensing device 2 in greater detail. FIG. 2 illustrates input and output signals for sensing device 2 implemented as a multiple axis MEMS accelerometer. As shown in FIG. 2, sensor 4 converts three axes of acceleration into three independent analog output voltages 22A-22C (collectively referred to as “analog output voltages 22”). Sensor circuitry 6 processes analog output voltages 22 to produce corresponding analog output voltages 23A-23C (collectively referred to as “analog output voltages 23”). Analog output voltages 23 represent the sensor signal generated by device 2 and provide posture and/or activity information that may be used by IMD 10 (not shown in FIG. 2). For example, as previously described, IMD 10 may examine analog output voltages 23 to confirm that a medical event was detected correctly.

In the example of FIG. 2, sensor 4 includes Z-axis accelerometer 20A and X-Y axis accelerometer 20B to measure acceleration along the three different axes. Each axis may be aligned with a different dimension of device 2. For example, the X-axis may be aligned along the length of device 2, the Y-axis may be aligned along the width of device 2, and the Z-axis may be aligned along the height of device 2. X-Y axis accelerometer 20B may be a single lateral accelerometer while Z-axis accelerometer 20A may be a separate differential teeter-totter accelerometer. Each of accelerometers 20A and 20B, however, may use differential capacitors to transduce acceleration into a corresponding output voltage. In this way, sensor 4 achieves a three-axis measurement using the combined X-Y axis accelerometer 20B to produce analog output voltages 22B and 22C and a separate Z-axis accelerometer 20A to produce analog output voltage 22A.

The interface between sensor circuitry 6 and sensor 4 may be fully differential and converts analog output voltages 22 into corresponding analog output voltages 23. As shown in FIG. 2, sensor circuitry 6 may include interface circuits 19A-19C (collectively referred to as “interface circuits 19”) that generate analog output voltages 23 from corresponding output voltages 22. In particular, interface circuits 19 may include circuit components to amplify, filter, and otherwise process signals 22 to produce stable, low noise output signals. For example, interface circuits 19 may include a low-power instrumentation amplifier with stable gain characteristics, good linearity, and wide common-mode range. The stable, low noise signals may then be examined by IMD 10 to determine the posture and/or activity of the patient. Thus, analog output voltages 23 may be used by IMD 10 to confirm that a medical event was detected correctly.

Sensor circuitry 6 may receive a control signal 28 that causes self-test circuitry 29 in device 2 to perform a self-test. As previously described, control signal 28 may be received in response to IMD 10 (not shown) detecting a therapy event, as a command from a programming device (not shown) associated with device 2 or IMD 10 (not shown), or during the manufacturing process for device 2. Self-test circuitry 29 may also apply a test signal, e.g., test signal 24A or test signal 24B, according to a schedule stored in local memory (not shown). In particular, self-test circuitry 29 may apply test signal 24A to interface circuits 19 for non-sensor activating self-test procedure, or test signal 24B to sensor 4 for a sensor-activating in response to receiving control signal 28.

Self-test circuitry 29 applies test signal 24A to interface circuits 19 without mechanically or otherwise activating sensor 4. Test signal 24A does not activate sensor 4. Instead, test signal 24A is selected to test the signal path from sensor 4 through sensor circuitry 6. The signal path may be characterized by extensive wire bonds that could become decoupled over time or in response to stress during use. Accordingly, self-test circuitry 29 may verify the continuity of the signal path for sensor 4 through interface circuits 19 by examining output voltages 23. For ease of illustration, not all of the signals are shown in FIG. 2. Specifically, FIG. 2 does not show output voltages 23 as being routed to self-test circuitry 29. If the test signal 24A applied to a given interface circuit 19 propagates through the signal path defined by the interface circuit, then self-test circuitry 29 may determine that sensing device 2 is operable, at least to the extent that there is no interconnection or component failure in sensor circuit 29.

Self-test circuitry 29 applies test signal 24B to sensor 4, thereby activating sensor 4. For ease of illustration, test signal 24B is not illustrated in FIG. 2 as being routed to each of accelerometers 20A and 20B. In this example, however, test signal 24B may be an electrostatic voltage that causes the beam fingers attached to the proof mass in accelerometers 20A and 20B to actuate, i.e., deflect along a particular direction. These deflections are translated to output voltages 22 and, thus, analog output voltages 23 which are examined by self-test circuitry 29. Self-test circuitry 29 determines the operational state of device 2 by comparing output voltages 23 to a predetermined voltage, range of voltages, or signal indicative of appropriate output of sensor 4 in response to test signal 24B.

The operational state of device 2 may be indicated by a verification signal 25 generated by sensor circuitry 6. The operational state of device 2 may be identified by varying the amplitude, frequency, or other parameter of verification signal 25. In this way, IMD 10 (not shown) receives verification signal 25 and determines if device 2 is functional or is not functional. IMD 10 may use verification signal 25 to verify whether the output of sensing device 2 can be used as a reliable trigger or validation or an event, or whether the output of the sensing device should be disregarded as unreliable due to malfunction of the sensing device.

Self-test circuitry 29 may also apply reset signal 27 to sensor 4. For example, self-test circuitry 29 may apply reset signal 27 to Z-axis accelerometer 20A, X-Y axis accelerometer 20B, or both when device 2 is determined to not be functional. As previously described, accelerometers 20A and 20B may fail to operate properly due to stiction. Thus, reset signal 27 may power accelerometers 20A and 20B on and off in an attempt to return the accelerometers to an operational state. Accordingly, reset signal 27 may be a signal that controls switches coupled to the input of accelerometers 20A and 20B.

In the example of FIG. 2, sensing device 2 and, more particularly, sensor circuitry 6, also may receive VDD, GND signals 21 and Trim/FLASH control signal 26. Generally, VDD, GND signals 21 provides power to various components of device 2. For example, VDD, GND signals 21 may supply reference and bias voltages to sensor 4 and sensor circuitry 6. Also, VDD, GND signals 21 may supply a nominal voltage for programming a trim memory register (not shown). A trim memory register may include electrically erasable programmable read only memory (EEPROM) cells or other cells of non-volatile memory that store trim calibration codes. In-package memory, e.g., EEPROM cells allow for device 2 to be calibrated on a high volume production line and then transferred for assembly, e.g., assembled as part of an IMD, such IMD 10 (not shown) that delivers therapy based at least partially on output generated by device 2. Trim/FLASH control signal 26 may be used to write calibration codes into the EEPROM cells of the trim memory register during assembly.

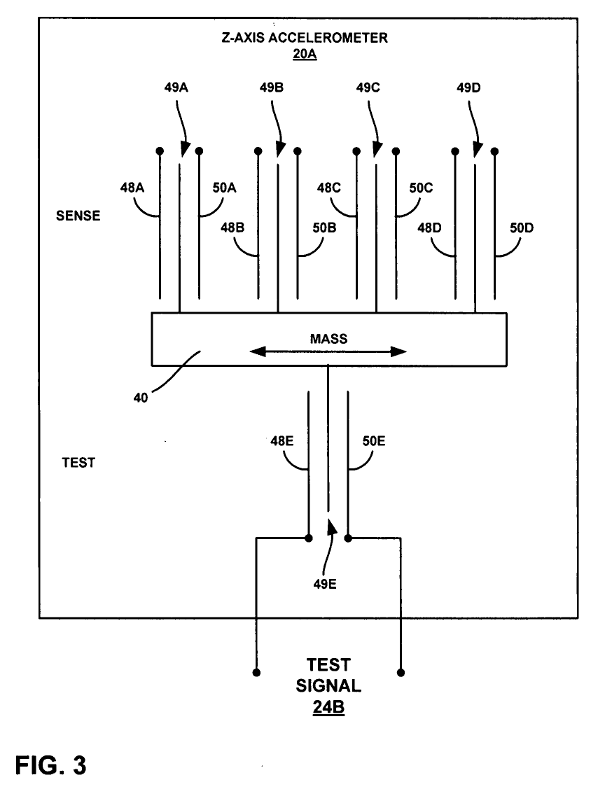

FIG. 3 is a block diagram illustrating an accelerometer channel 20A for use in sensing device 2 of FIG. 2. In the example of FIG. 3, Z-axis accelerometer channel 20A operates as previously described with respect to FIG. 2 and may be constructed as a single-axis MEMS accelerometer. Z-axis accelerometer 20A includes a plurality of beam fingers 49A-49D attached to proof mass 40. Proof mass 40 may be suspended over a substrate by one or more springs coupled to an inertial frame. Beam fingers 49 are inter-digitated with fixed fingers 48A-48D and fixed fingers 50A-50D. For example, beam finger 49A is positioned between fixed fingers 48A and 50A, beam finger 49B is positioned between fixed fingers 48B and 50B, and so forth. Fixed fingers 48, 50 may be fixed directly to a substrate or may be attached to an inertial frame that is fixed to the substrate.

Fixed fingers 48, 50 interact with beam fingers 49 to form variable, differential capacitors. More specifically, in FIG. 3, fixed fingers 48A-48D form a variable capacitor in combination with beam fingers 49A-49D. Similarly, fixed fingers 50A-50D form a variable capacitor in combination with beam fingers 49A-49D. Beam fingers 49A-49B are electrically coupled to one another and receive an excitation signal. Fixed fingers 48A-48D are electrically coupled to one another and form a positive input to a differential amplifier in sensor circuitry 6. Fixed fingers 50A-50D are electrically coupled to one another and form a negative input to a differential amplifier in sensor circuitry 6.

As proof mass 40 is deflected in a particular direction (indicated by the arrow in FIG. 3), the capacitance measured between one of beam fingers 49 and one of the corresponding fixed fingers 48 or 50 increases and the capacitance measured between the beam finger and the other corresponding fixed finger 48 or 50 decreases for the same direction of motion. For example, when mass 40 is deflected to the left in FIG. 3, the capacitance between beam finger 49A and fixed finger 48A increases and the capacitance between beam finger 49A and fixed finger 50A decreases, given the inverse relationship of capacitance versus distance between capacitive plates.

As fixed fingers 48A-48D are coupled together, the resulting variable capacitance between fixed fingers 48A-48D and beam fingers 49A-49D is additive among the fingers. The same is true for fixed fingers 50A-50D. In this manner, when an excitation signal is applied to beam filter 49A, the potentials at fixed finger 48A and fixed finger 50A vary according to the deflection of mass 40 and form a differential voltage indicating the amount of deflection. The differential voltage generated across fixed fingers 48A-48D and 50A-50D is coupled to a differential amplifier in interface circuit 19A of sensor circuitry 6, and is represented by signal 22A in FIG. 2. X-Y axis accelerometer channel 20B may have a similar arrangement, but may include proof masses and associated beam fingers and fixed fingers arranged in lateral X and Y directions to produce differential voltages 22B, 22C representing displacement in those directions. Interface circuit 19A may amplify and filter the output voltage 22A and produce a corresponding sensor output voltage 23A for use by IMD 10.

As further shown in FIG. 3, Z-axis accelerometer channel 20A may have a sense side and a test area. As described above, various beam fingers 49 and fixed fingers 48, 50 may be arranged to produce a differential voltage indicative of displacement of proof mass 40. However, to support a self-test feature within Z-axis accelerometer channel 20A, at least one additional beam finger 49E and fixed fingers 48E, 50E may be provided. In particular, sensor circuitry 6 may apply the test signal 24B as an electrostatic voltage across fixed fingers 48E and 50E.

The electrostatic voltage causes beam finger 49E to deflect, in turn causing proof mass 40 and beam fingers 49A-49D to deflect. The test signal may be selected to cause deflection by a known amount. On this basis, sensor circuitry 6 monitors the differential voltage output of Z-axis accelerometer channel 20A to verify operation of the Z-axis accelerometer. A similar arrangement may be provided for X-Y accelerometer channel 20B such that the test signal can be applied to cause a known amount of deflection in the X and Y directions, and thereby verify proper operation of X-Y accelerometer channel 20B.

The reset function may be performed by toggling the reset signal 27 on and off. The reset signal 27 may be coupled to a switch that couples and decouples the reference or excitation voltage to and from beam fingers 49A-49D. Alternatively, the reset signal 27 may be the reference or excitation voltage. In either case, the voltage applied across the variable capacitors formed by fingers 48 and 49, and by fingers 49 and 50, is turned on and off, releasing attractive forces that may be causing stiction between the fixed fingers and the beam fingers. In this manner, reset signal 27 may be applied by sensor circuitry 6 to restore operation of sensor 2, particularly in the case of an accelerometer or other capacitive-based sensing device.

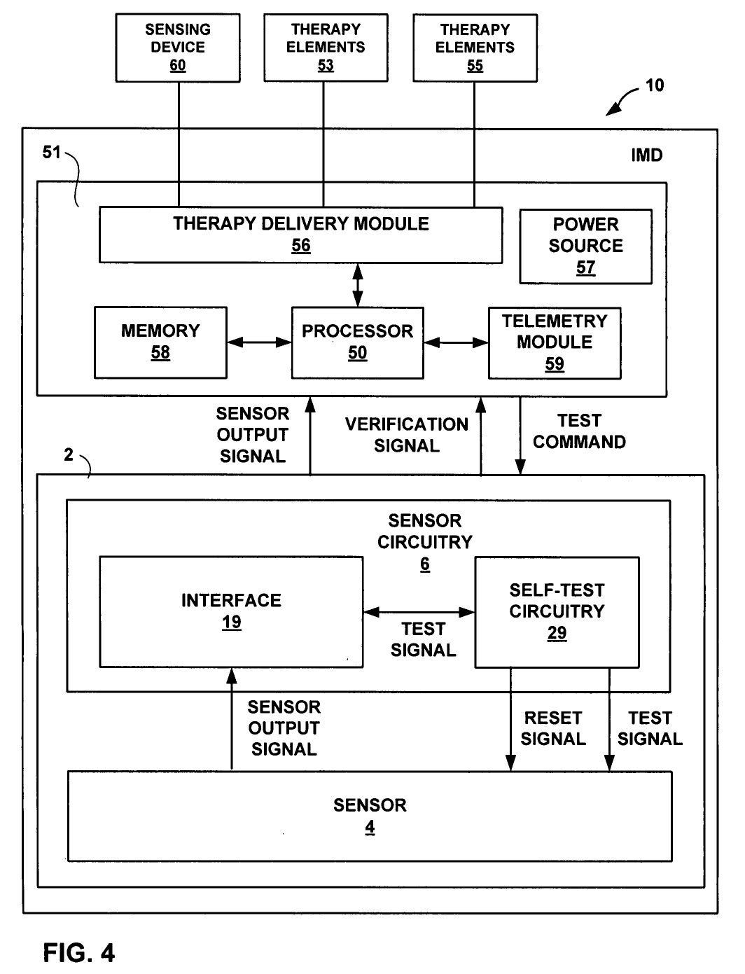

FIG. 4 is a block diagram illustrating an example IMD 10 including a therapy delivery device 51 and sensing device 2. IMD 10 is implantable within a patient to deliver therapy and/or record sensed data for diagnostic purposes. The therapy may be at least partially based on output provided by sensing device 2. Sensing device 2 includes the previously described self-test features that enable device 2 to verify that it is functional while implanted within the patient. In this way, IMD 10 may deliver therapy to the patient with increased confidence when a medical event that influences a therapy or diagnostic adjustment is either triggered or validated by sensing device 2.

In the example of FIG. 4, therapy delivery device 51 includes therapy delivery module 56, processor 50, memory 58, power source 57, and telemetry module 59. Therapy delivery module 56 may deliver therapy in the form of electrical pulses, one or more drugs, or a combination of both via at least one of therapy elements 53 and 55. As examples, IMD 10 may be configured as an implantable neurostimulator, cardioverter/defibrillator, or drug delivery device. Alternatively, or additionally, IMD 10 may operate as a diagnostic device, such as a loop recorder. For electrical stimulation, each therapy element 53 and 55 may include one or more electrodes carried on one or more leads or carried on the housing of IMD 10. In this case, therapy delivery module 56 may include an implantable stimulation generator or other stimulation circuitry that generates electrical stimulation waveforms, such as stimulation pulses or continuous signals under the control of processor 50. Alternatively, therapy elements 53 and 55 may include one or more fluid delivery devices such as catheters.

Therapy delivery module 56, processor 50, telemetry module 59, and memory 58, receive operating power from power source 57. Power source 57 may take the form of a small, rechargeable or non-rechargeable battery, or an inductive power interface that transcutaneously receives inductively coupled energy. In the case of a rechargeable battery, power source 57 similarly may include an inductive power interface for transcutaneous transfer of recharge power from a charging device outside of the patient's body.

In embodiments in which one or more fluid delivery devices form part of therapy elements 53 and 55, therapy delivery module 56 may include one or more fluid reservoirs and one or more pump units that pump fluid from the fluid reservoirs to the target site through the fluid delivery devices. The fluid reservoirs may contain a drug or mixture of drugs. The fluid reservoirs may provide access for filling, e.g., by percutaneous injection of fluid via a self-sealing injection port. The fluid delivery devices may comprise, for example, catheters that deliver, i.e., infuse or disperse, drugs from the fluid reservoirs to the same or different target sites.

Processor 50 may include a microprocessor, microcontroller, digital signal processor (DSP), application specific integrated circuit (ASIC), field programmable gate array (FPGA), discrete logic circuitry, or a combination of such components. Processor 50 may be programmed to control delivery of therapy according to selected parameter sets stored in memory 58. The parameter sets stored in memory 58 may specify amplitudes, pulse widths, frequency, and/or electrode polarities for stimulation therapy. In the case of drug therapy, the parameter sets stored in memory 58 may include dosage, rate and limit parameters for drug delivery. In addition to programs, memory,58 may also store schedules for delivering therapy to the patient. Memory 58 may include any combination of volatile, non-volatile, removable, magnetic, optical, or solid state media, such as read-only memory (ROM), random access memory (RAM), electronically-erasable programmable ROM (EEPROM), flash memory, or the like.

Telemetry module 59 may allow processor 50 to communicate with an external programmer, such as a clinician programmer or patient programmer, or with another implanted device such as an implanted sensor or therapy device. Notably, in some embodiments, sensing device 2 may be separately implanted and configured to communicate with IMD 10 or an external programmer via wireless telemetry. Processor 50 may receive programs defining parameters for delivery of therapy to a patient from external programmer via telemetry module 59 during programming by a clinician. Where therapy delivery device 10 stores parameter sets in memory 58, processor 50 may receive parameter sets from the clinician programmer via telemetry module 59 during programming by a clinician, and later receive parameter set selections made by the patient from the patient programmer via telemetry module 59. If the programmer stores the parameter sets, processor 50 may receive parameter sets selected by patient from programmer via telemetry module 59. In addition, processor 50 may receive parameter adjustments from the external programmer.

In general, sensing device 2 may operate as previously described in this disclosure and includes sensor 4 and sensor circuitry 6. Accordingly, sensor 4 may comprise any sensor capable of sensing a physiological parameter of the patient in which IMD 10 is implanted. Thus, sensor 4 outputs one or more signals that are indicative of the sensed parameter to sensor circuitry 6. As an example, sensor 4 may be a three-axis MEMS accelerometer configured to operate as previously described in this disclosure. In this case, sensor 4 may generate analog output voltages, such as output voltages 22, that are indicative of motion or force in different dimensions and can be used to indicate patient posture and/or activity.

In the example of FIG. 4, sensor circuitry 6 includes self test circuitry 29 and interface circuit 19. If appropriate, interface circuit 19 may include separate interface circuits 19A, 19B, 19C for different sensor channels as shown in FIG. 2. Interface 19 provides a signal path for the output of sensor 4, and may include components for amplification, filtering and/or other processing of the sensor output signal to produce a stable, low noise signal. As shown in FIG. 4, self-test circuitry 29 may apply a test signal to interface 19 to verify sensor operation without activating sensor 4, or apply a test signal to sensor 4 to activate the sensor to verify sensor operation. In addition, self-test circuitry 29 may generate a reset signal to reset sensor 4 if the test signal indicates that the sensor is not operable.

Therapy delivery device 51 of IMD 10 receives the sensor output signal from sensing device 2, and may use the sensor output signal to trigger or validate medical events that drive adjustment of therapy of diagnostic operations within the IMD, as described in this disclosure. As an example, if IMD 10 is a neurostimulator, and the sensor output signal from sensing device 2 indicates a posture or activity change, the sensor output signal may trigger a medical event that drives a therapy adjustment. In this case, therapy delivery device 51 may issue a test command to sensing device 2 in response to the detected event to initiate a self-test by self-test circuitry 29. If the self test indicates that sensing device 2 is operable, therapy delivery device 51 may then proceed with the therapy adjustment. Alternatively, in some embodiments, self-test circuitry 29 may autonomously initiate the self-test based on detection of a sensor output signal that triggers medical event by sensor circuitry 6.

In any event, therapy delivery device 51 may identify an event if the sensor output signal has a characteristic associated with an event, such as an amplitude level, frequency, trend, or other characteristic. In the case of a posture change, the sensor output signal may indicate an accelerometer displacement along one or more axes that correlates with a particular posture or a change in posture. If therapy delivery device 51 verifies sensor operation via self-test circuitry 29, then therapy adjustment can be made with better confidence. In this case, therapy delivery device 51 determines that therapy adjustment is appropriate and not falsely triggered by a malfunctioning sensor 2. On this basis, therapy delivery device 51 proceed with therapy adjustment.

Similarly, therapy delivery device 51 may use sensor 2 to validate an event sensed by a different sensor, e.g., by determining that the output of sensor 2 is consistent with the event. In this case, therapy delivery device 51 may request application of the self-test feature to ensure that sensing device 2 is operable. The self-test feature may be applied whenever sensing device 2 is called upon to validate an event sensed by another sensing device, such as sensing device 60 of FIG. 4. Alternatively, in some embodiments, the self-test feature may be activated if sensing device 2 generates a sensor output signal that does not correlate, and is therefore inconsistent, with the event sensed by sensing device 60. If sensing device 2 is found to be operable and generates output correlates with the event sensed by sensing device 60, then the event is validated. In this case, therapy delivery device 51 may proceed with a therapy or diagnostic adjustment.

If sensing device 2 is found to be operable and generates output that does not correlate with the event sensed by the other sensing device 60, therapy delivery device 51 may be configured to withhold the therapy or diagnostic adjustment or apply further analysis. If sensing device 2 is found to be inoperable, its output may be disregarded for purposes of validating the event, and the inoperability may be notified or recorded for the attention of a medical caregiver. Hence, with the self-test feature, therapy delivery device 51 can determine whether a lack of correlation or consistency between the event and the output of sensing device 2 is due to a malfunctioning sensing device 2 and, in the event of correlation, proceed with therapy or diagnostic adjustment with a greater degree of confidence.

As an example, IMD 10 may be a cardiac stimulation device, and sensing device 60 may be formed by a set of ECG sense electrodes. In this case, processor 50 may analyze signals sensed by sensor 60 to identify cardiac arrhythmia or fibrillation. If processor 50 identifies cardiac fibrillation, IMD 10 may use sensing device 2, which may be an accelerometer, to validate the identified fibrillation. For example, IMD 10 may analyze the sensor output signal from sensing device 2 to determine whether slumped over, laying down, inactive, or experienced a sudden fall. If a sudden fall is indicated, the sensor output signal from sensing device 2 correlates with the fibrillation indicated by sensing device 60. In this case, IMD 10 may proceed with delivery of therapy to the patient, such as a defibrillation shock. If the sensor output signal from sensing device 2 does not correlate with the fibrillation, however, IMD 10 may withhold delivery of a shock pending further analysis of the output of sensing device 60 or other sensing devices.

In this manner, sensing device 2 provides a sensor output signal to validate or invalidate the fibrillation event sensed by sensing device 60. Hence, if the output of interface 19 indicates that the patient is likely to be experiencing an arrhythmia, such as fibrillation, therapy delivery device 51 may deliver therapy to the patient. However, if the output does not indicate that the patient is likely to be experiencing an arrhythmia, e.g., by detecting that the patient is walking or standing, therapy delivery device 51 may determine that the fibrillation was detected incorrectly and withhold therapy, or at least initiate further analysis. In either case, therapy delivery device 51 may issue a test command in response to the detected event that causes self-test circuitry 29 to test sensing device 2, either by activating sensor 4 or testing the integrity of interface 19.

To increase the confidence with which therapy delivery device 51 delivers or withholds therapy, self-test circuitry 29 performs a self-test to verify that sensing device 2 is operational. In some embodiments, self-test circuitry 29 may include test signal generator, memory, and a timer (not shown). In one example, self-test circuitry 29 may perform a self-test periodically, such as in accordance with a maintenance schedule stored in the memory. The timer may track the maintenance schedule information in the memory to cause self-test circuitry 29 to initiate periodic self-tests in accordance with one or more schedules.

As an example, the memory may store one or more values and the timer may load a value from the memory. The values stored in the memory may correspond to values that result in more or less frequent self-tests. In some embodiments, it may be desirable to perform self-tests more frequently, such as in life sustaining applications, so as to ensure that sensing device 2 is functional more frequently. However, in other embodiments, it may be desirable to perform self-tests less frequently in order to conserve power. In some embodiments, the schedule may be selectable to provide a “normal” mode that performs self-tests at desired time intervals, and a “low power” mode that performs self-tests less frequently. The timer may provide a countdown timer with a reset. In operation, the timer may load a value from the memory under the control of a processor. The timer may then count down from the loaded value until the counter expires. When the counter expires, the timer may cause self-test circuitry 29 to perform a self-test and reset itself.

In some embodiments, self-test circuitry 29 may include a processor that controls self-test circuitry 29 to perform a self-test. Specifically, the processor may cause self-test circuitry 20 to perform a self-test on command, as a verification step in a therapy or diagnostic adjustment algorithm, or during a manufacturing process. For example, the processor may cause self-test circuitry 29 to perform a self-test in response to receiving a command from a programming device, such as a patient or clinician programmer associated with IMD 10. The command may be generated in response to input entered into the programming device by a user, such as manual depression of one or more keys of the programming device. IMD 10 receives the command, for example via telemetry module 59, and directs a test command to self-test circuitry 29. In this case, telemetry module 59 may receive the command and processor 50 may route the command to a self-test circuitry or a processor associated with self-test circuitry. In response, self-test circuitry 29 performs a self-test of sensing device 2 in accordance with the received command. The self-test may be a sensor activation test, a non-activation test of interface 19, or both.

When self-test circuitry 29 performs a self-test as a verification step of a therapy delivery algorithm, self-test circuitry 29 may receive a control signal from therapy delivery device 51. For example, processor 50 may transmit a control signal to self-test circuitry 29 as a test command in response to detecting an event based on output from sensing device 2 or output from another sensing device 60. In this case in which the event was detected by another sensing device 60, therapy delivery device 51 relies on sensing device 2 to provide information that confirms that the event was correctly detected.

Self-test circuitry 29 may perform a self-test during a manufacturing process to verify that the device is operational. In this case, a control signal may be applied to processor self-test circuitry. Alternatively, a test signal may be applied directly to sensor 4 or sensor circuitry 6 during the manufacturing process, for example, via self test pins located on sensing device 2. In any case, the self-test may streamline the manufacturing process by replacing more costly and time consuming physical testing.

In addition to controlling when device 2 performs a self-test, sensing circuitry 29 controls how the self-test is performed. Again, self-test circuitry 29 may perform a self-test by activating sensor 4 or without activating sensor 4. Self-test circuitry 29 performs a self-test that activates sensor 4 by applying a test signal to sensor 4 that causes sensor 4 to generate an output signal. Self-test circuitry 60 performs a self-test without activating sensor 4 by analyzing the continuity of the signal path for sensor 4, e.g., by applying a test signal directly to interface 19 without mechanically or otherwise activating sensor 4. In some embodiments, sensor circuitry 6 may include both activating and non-activating self-test features, which may be applied individually or together. Alternatively, sensor circuitry 6 may include only one type of self-test feature, i.e., activating or non-activating but not both.

Self-test circuitry 29 may include a test signal generator (not shown) that generates the test signal for self-tests that activate sensor 4 and self-tests that do not activate sensor 4. The test signal generator may include a charge pump that generates the test signal as an electrostatic voltage for an activating self-test of sensor 4, and/or a signal generator that generates a test signal as an electrical signal/waveform for non-activating self-test of interface 19. When self-test circuitry 29 performs an activating self-test, the test signal generator activates sensor 4 by applying an electrostatic voltage to sensor 4, thereby causing sensor 4 to generate an output.

For example, when sensor 4 is implemented as a three-axis MEMS accelerometer, the test signal generator associated with self-test circuitry 29 may apply an electrostatic voltage to one or more fingers of each accelerometer. With respect to FIG. 3, the electrostatic voltage may be applied to one or more of fixed fingers, such as fixed fingers 48E, 50E. The electrostatic voltages actuates the beam finger 49E, and deflects the beam finger in a particular direction, causing sensor to generate a sensor output signal. Self-test circuitry may analyze the sensor output signal for each axis of the accelerometer in response to the electrostatic test voltage to verify proper operation of sensor 4.

A processor or analog comparator circuitry associated with self-test circuitry 29 may examine the sensor output signal to determine the operational state of sensor 4. If a processor is used, self-test circuitry 29 may include and analog-to-digital converter. The sensor output signal may be compared to a threshold value or range of values stored in memory. Self-test circuitry 29 may generate a verification signal based on the comparison. The verification signal may indicate the operational state of device 2, and may be transmitted to therapy delivery device 51. The verification signal may simply indicate either operability or inoperability of sensing device 2. Alternatively, the verification signal may provide more detailed operational information, such as a performance level of sensing device 2. As a further example, for a multi-axis accelerometer, the verification signal may indicate which axes are working and which axes are not working. Therapy delivery device 51 may use the verification signal as part of a therapy or diagnostic adjustment algorithm as described in this disclosure.

To perform a non-sensor activating self-test, a test signal generator associated with self-test circuitry 29 may apply a test signal to interface circuitry 19 in the form of an electrical signal/waveform. The electrical signal/waveform may be a constant reference voltage or a waveform. The electrical signal/waveform does not mechanically or otherwise activate sensor 4. In this case, the electrical signal/waveform may be a model of a waveform, such as a cardiac waveform, a sinusoidal waveform, a square wave, or the like. When the test signal is a model of a waveform, the test signal may be generated from example waveforms. For example, the test signal may be generated as an average of two or more example waveforms. Alternatively, the electrical signal/waveform may be a constant reference voltage.

The continuity of the signal path through interface 19 can be analyzed applying the test signal to the interface input and analyzing the interface output. In particular, self-test circuitry 29 examines the output of interface 19 in response to the test signal to determine if the test signal or some amplified, filtered, or otherwise processed signal successfully propagated through interface 19. If there is no signal at the output of the interface 19, or the signal at the output is not what is expected in response to the test signal at the input, interface 19 may be deemed inoperable by self-test circuitry 29. If the signal successfully propagates through interface 19, electrically continuity and functionality of sensing device 2 is verified. If not, interface 19 is inoperable, e.g., to a bond or component failure. On this basis, self-test circuitry 29 indicates in the verification signal that sensing device 2 is non-functional.

If self-test circuitry 29 includes a processor, it may be formed, like processor 50, from one or more microprocessors, DSPs, ASICs, FPGAs, other discrete or integrated logic circuitry, or any combination of such components. Any memory associated with self-test circuitry 29 may be implemented, like memory 58 of therapy delivery device 51, as a single memory module or physically separate memory modules and may include any combination of volatile, non-volatile, removable, magnetic, optical, or solid state media, such as read-only memory (ROM), random access memory (RAM), electronically-erasable programmable ROM (EEPROM), flash memory, or the like. In some embodiments, sensor circuitry 6 also may include memory that stores calibration trim codes for sensor 4 that are written to memory during production.

In the example of FIG. 4, sensing device 2 is contained within IMD 10, e.g., as an accelerometer mounted within an IMD housing. However, at least a portion of sensing device 2 may alternatively be located outside of IMD 10. For example, sensing device 2 may be located on a housing of IMD 10. In another example, sensing device 2 may be located within a lead, such as located at a distal tip of a lead, extending from IMD 10 and electrically coupled to the IMD via conductors. In any case, sensing device 2 communicates with therapy delivery device 51 via a wired or wireless connection. In exemplary embodiments, sensing device 2 may be soldered or otherwise directly electrically connected to circuitry within therapy delivery device 51, such as via wire bonds. In either case, sensing device 2 provides information in the form of electrical signals to therapy delivery device 51.

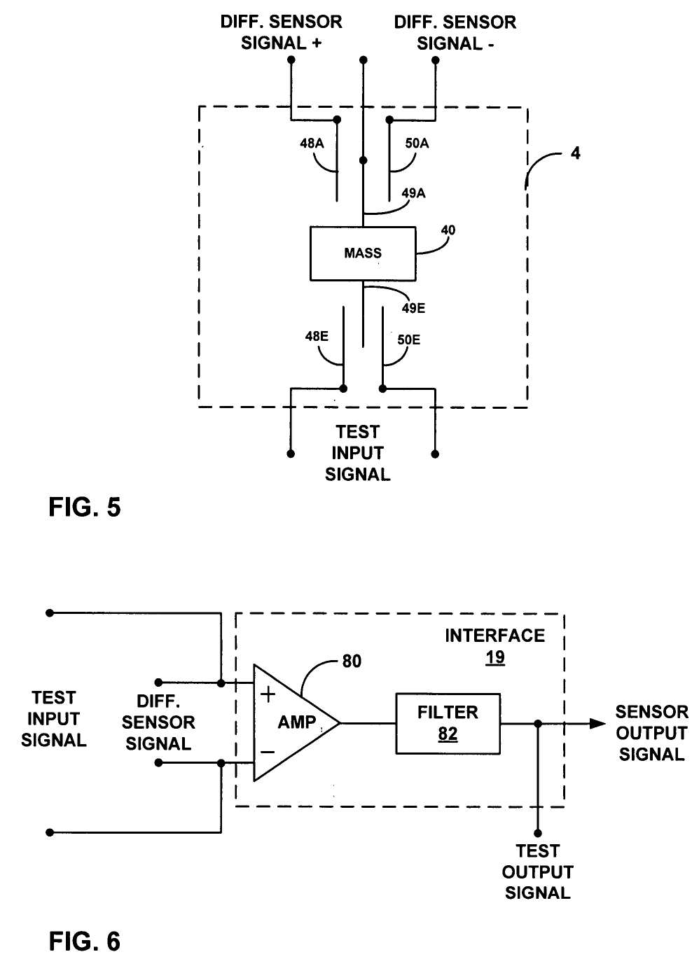

FIG. 5 is a block diagram illustrating a self-test feature that applies a test signal to activate a sensor 4 in a sensing device 2. In the example of FIG. 5, sensing device 2 is an accelerometer. Only one axis of the accelerometer is shown for ease of illustration. Sensing device 2 conforms substantially to the accelerometer of FIG. 3. However, only a single set of fixed fingers 48A, 50A and beam finger 49A is shown for ease of illustration. As shown in FIG. 5, fixed finger 48A may be coupled to the positive input of a differential instrumentation amplifier to form a positive differential sensor signal (DIFF. SENSOR SIGNAL +). Similarly, fixed finger 50A may be coupled to the negative input of a differential instrumentation amplifier to form a negative differential sensor signal (DIFF. SENSOR SIGNAL −).

As described with respect to FIG. 3, the differential signal generated by the variable capacitance between fixed finger 48A and beam finger 49A and between fixed finger 50A and beam finger 49A is a function of deflection of proof mass 40, coupled to beam finger 49A. To test sensor 4, sensor self-test circuitry 29 may apply a test input signal as an electrostatic across fixed fingers 48E and 50E, which causes beam finger 49E to deflect. Deflection of beam finger 49E causes proof mass 40 to deflect, resulting in a change in the accelerometer output signal at fingers 48A, 50A. The change in the accelerometer output signal can be compared to an expected accelerometer output signal in response to the deflection caused by the test input signal to determine proper operability of sensor 4.

FIG. 6 is a block diagram illustrating a self-test feature that applies a test signal to an interface 19 to verify interface integrity without activating a sensor 4 in a sensing device 2. In the example of FIG. 6, interface 19 includes a differential amplifier 80 and filter 82. However, interface 19 may include additional components or different components. In operation, interface 19 receives a differential sensor signal from sensor 4 at the differential inputs of amplifier 80, and amplifies and filters the signal to produce a sensor output signal, e.g., for use by therapy delivery device 51 of IMD 10. To test the integrity of interface 19, self-test circuitry 29 applies a test input signal at the differential input of amplifier 80 and monitors the sensor output signal as a test output signal in response to the test input signal. Again, the test input signal may be a constant reference voltage or a varying signal waveform. In either case, self-test circuitry 29 compares the test output signal to an expected output signal to verify whether interface 19 is operable or not.

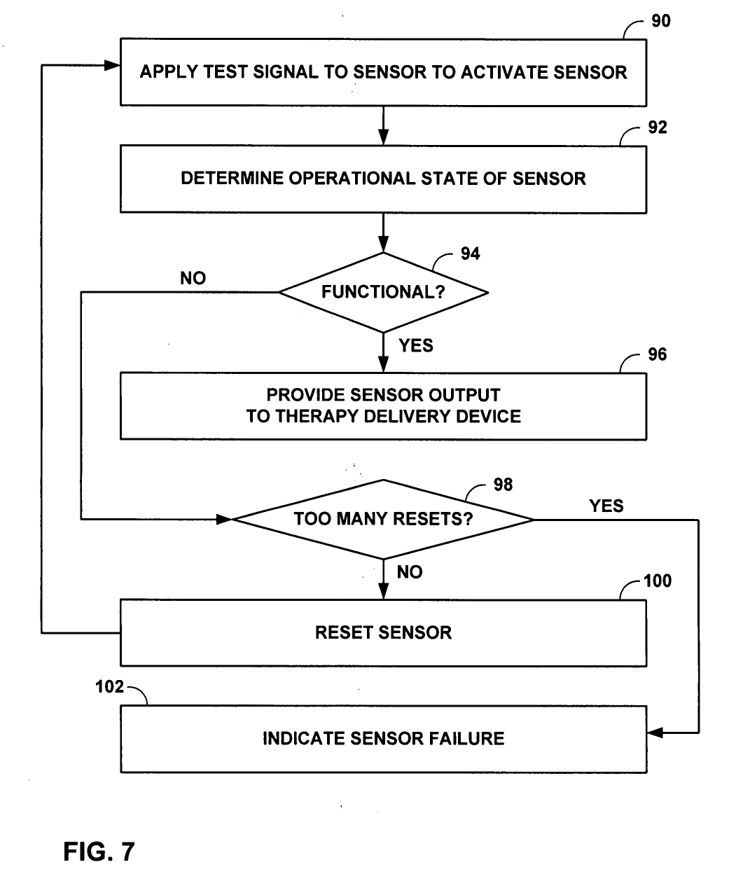

FIG. 7 is a flow diagram illustrating an example mode of operation of sensing device 2 for performing a self-test that activates sensor 4. The self-test begins by applying a test signal to sensor 4 to activate sensor 4 (90). As previously described, sensing device 2 and, more particularly, sensor circuitry 6, may activate sensor 4 by applying the test signal in accordance with a schedule, in response to a command, as a verification step in a therapy or diagnostic adjustment algorithm associated with an IMD, or as a part of the manufacturing process for device 2.

The test signal may be generated by self-test circuitry 29 in sensor circuitry 6. The test signal activates sensor 4 in the sense that the test signal causes sensor 4 to generate an output signal. Using an accelerometer, such as a MEMS accelerometer, as an example, the test signal may cause capacitive plates, i.e., one or more beam fingers, to actuate. The test signal in this case may be an electrostatic voltage applied to one or more fixed fingers. As a result, sensor circuitry 6 may determine the operational state of sensor 4 (92) based on the output generated by sensor 4 in response to the test signal. In other words, sensing device 2 verifies that it is functional based on the output of sensor 4 in response to the test signal. Sensor circuitry 6 may determine the operational state of sensor 4, for example, by comparing the output to a predetermined threshold value or range of values that would be expected if sensor 4 were operable.

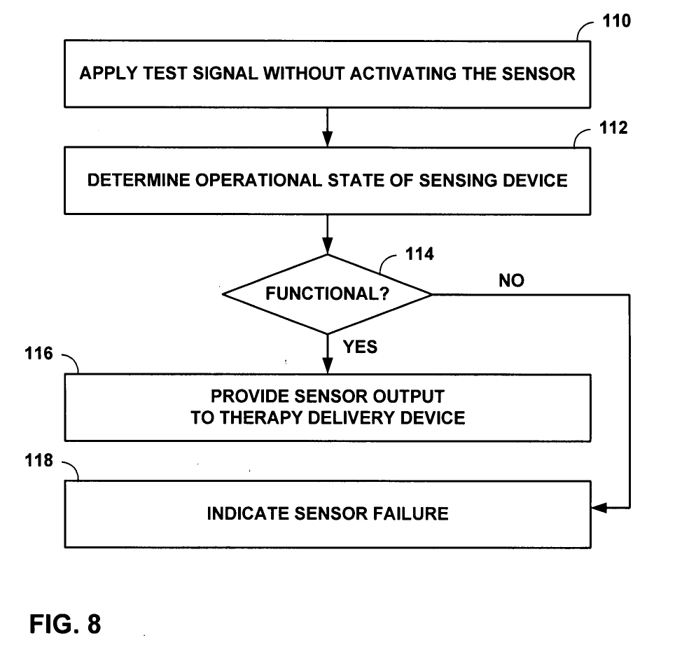

When sensor 4 is determined to be functional (“YES” branch of decision block 94), sensing device 2 provides the output to a therapy delivery device (96), such as therapy delivery device 51 in FIG. 4. The therapy delivery device may then deliver therapy to a patient based at least partially on the output, as previously described in this disclosure. An example process for using the output in a therapy delivery algorithm is provided in FIG. 8. If sensing device 2 is determined to not be functional (“NO” branch of decision block 94), sensing device 2 uses a reset feature to attempt to return itself to a functional state. Sensing device 2 may attempt to reset itself one or more times in an attempt to return itself to a functional state. Accordingly, in the flow diagram shown in FIG. 7, sensing device 2 first determines if it has reset itself too many times (decision block 98). This serves as an end condition so that the sensing device 2 does not continue to reset itself.

In the case that sensor 4 is not operational (“NO” branch of decision block 94), sensor circuitry 6 may determine if sensor 4 has been reset too many times (decision block 98). If sensor 4 has not been reset too many times (“NO” branch of decision block 98), sensor circuitry 6 resets sensor 4 (100), for example, by powering sensor 4 on and off with a reset signal. Powering sensor 4 on and off may result in returning sensor 4 to a functional state in some cases, such as when sensor 4 becomes inoperable due to stiction. In the event that a reset does return to a functional state, steps 90, 92, 94, and 96 are repeated and the flow ends in providing sensor output to therapy delivery device 51 (96).

However, if the self-test test continues to result in resetting the accelerometer, the maximum number of resets allowed will be reached (“YES” branch of decision block 98) and sensing device 2 indicates to an operator that the accelerometer has failed (102). Sensing device 2 may indicate this by, for example, communicating with therapy delivery device 51 to telemeter this information to an external programmer. The external programmer may activate an appropriate indicator light or display a message to an operator via a user interface in response to receiving the signal from sensing device 2 or therapy delivery device 51. It is also conceivable that sensing device 2 or therapy delivery device 51 may provide an indication detectable by the patient, such as a vibration or audible alert. As a further option or alternative, the sensor malfunction may be recorded in memory for later analysis by a medical care-giver.

FIG. 7 depicts a self-test process that is applied to determine whether a sensor 4 is operable. If the sensor 4 is found to be operable, sensor output signals generated by the sensor are provided to therapy delivery device 51. In this sense, the process of FIG. 7 may verify sensor operation before providing a sensor output signal to therapy delivery device 51. However, the self-test process may be applied after a sensor output signal is provided to therapy delivery device 51, or applied periodically or on-demand during continuous or periodic delivery of sensor output signals to therapy delivery device 51. As one example, sensing device 2 may be coupled to provide sensor output signals to therapy delivery device 51 on an ongoing basis. When therapy delivery device 51 detects an event that drives adjustment of therapy or diagnostic operations based on a sensor output signal from sensing device 2, therapy delivery device 51 may issue a test command to cause self-test circuitry 29 to perform a self-test of sensing device 2 and thereby verify proper operation before proceeding with a therapy or diagnostic adjustment based on the sensor output signal.

FIG. 8 is a flow diagram illustrating an example mode of operation of sensing device 2 for performing a self-test that does not activate sensor 4. In general, the process shown in FIG. 8 can be used to verify continuity of the signal path of an interface associated with sensor 4 during manufacturing or post implant. This may be particularly useful for streamlining the manufacturing process, but also useful following implantation of sensor 4. The process of FIG. 8 may be carried out to test a sensor without performing costly tests that activate sensor 4, such as shake tests for an accelerometer, during manufacturing, or without activating sensor 4 by application of an electrostatic voltage. This non-activating self-test may be applied alone or in conjunction with a sensor activating self-test.

The self-test begins by applying a test signal without activating sensor 4 (110). With respect to FIG. 8, self-test module 60 may apply the test signal directly to an input of interface 19 without mechanically or otherwise activating sensor 4. In this case, the test signal may be an electrical signal or waveform, such as a sinusoidal waveform, square wave, or the like. Alternatively, the electrical signal may be a model generated from one or more example waveforms. For example, the test signal may be generated as an average of multiple example signals. As a further alternative, the test signal may be a constant reference voltage.

Self-test circuitry 29 may determine the operational state of sensing device 2 (112), for example, by determining if the test signal, or some amplified, filtered, or processed version of the signal has propagated to the output of interface 19. This may be accomplished by comparing the output of interface 19 to an example waveform or a threshold value or range of values. If a signal successfully propagates through interface 19, i.e., the output of interface 19 produces a signal having expected characteristics, self-test circuitry 29 determines that sensing device 2 is functional (“YES” branch of decision block 114), i.e., electrical continuity and circuit component operability is confirmed. In this case, sensing device 2 may provide sensor output to therapy delivery device 51 (step 116).

If the signal does not successfully propagate through interface 19, sensing device 2 determines that interface circuitry 19 has failed, resulting in inoperability of sensor device 2. Accordingly, sensing device 2 indicates to an operator that device 2 has failed (118). Again, as in the example of FIG. 7, the indication of a failure in FIG. 8 may be recorded in memory or notified to a user, such as a medical caregiver or patient, via a variety of techniques.

As in the example of FIG. 7, the process of FIG. 8 may verify sensor operation before providing a sensor output signal to therapy delivery device 51, or after a sensor output signal is provided to therapy delivery device 51. Also, in some embodiments, non-activating sensor self-test may be applied on a tiered basis with an activating sensor self-test. For example, sensor self-test circuitry 29 may first apply the activating self-test to sensor 4. If the self-test does not result in an appropriate output signal, sensor self-test circuitry 29 may then apply a non-activating self-test to interface 19 to determine whether the sensor malfunction was caused by sensor 2, interface 19, or both.

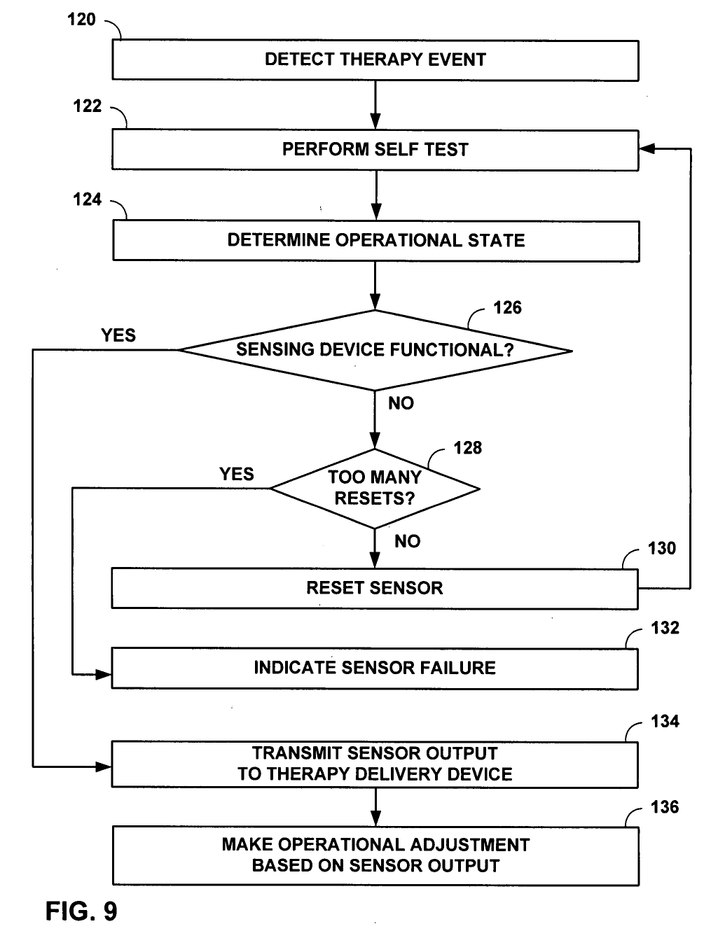

FIG. 9 is a flow diagram illustrating an example technique for using sensing device 2 to provide key verification information in the course of a therapy or diagnostic adjustment algorithm, which support an important, or even life sustaining, therapy application. In this example, therapy delivery device 51 delivers therapy based on the output of sensor 4. Without performing a self-test, the therapy is delivered with a level of uncertainty because it is unknown if sensing device 2 is functional at any given time. However, by performing a self-test that verifies device 2 is operational, the therapy can be delivered with a lesser degree of uncertainty.

The technique begins in FIG. 8 by detecting a therapy event (120). The therapy event may be detected by therapy delivery device 51 based on output from a self-test sensing device 2 or output from a different sensor. For example, therapy delivery device 51 may detect a therapy event based on the output of self-test sensing device 2, or detect a therapy event based on the output of a different sensing device. Self-test sensing device 2 may be used as a primary trigger to initiate, terminate or control therapy, each of which may be considered a therapy adjustment. Also, self-testing device 2 may be used as a primary trigger for a diagnostic adjustment, such as activation of a loop recording, or adjustment of sampling frequency or sensitivity for loop recording. As mentioned previously, therapy or diagnostic adjustments may be generally referred to as operational IMD adjustments.

As an alternative, self-test sensing device 2 may be used as a secondary trigger to validate a primary trigger of an event by another sensing device. In this latter case, therapy delivery device 51 may adjust therapy or diagnostic operation if the output of self-test sensing device 2 correlates with the event triggered by the other device, withhold adjustment of therapy or diagnostic operation if the output of self-test sensing device 2 does not correlate with the therapy event, or withhold adjustment of therapy or diagnostic operation pending further analysis if the output of self-test sensing device 2 does not correlate with the therapy event.

The therapy event, which may be detected based on output from self-test sensing device 2 or a different device, as mentioned above, may be any of a variety of sensed events. Sensed therapy events may be sensed by electrical, electromechanical, chemical, biological, or other sensors. Examples of sensed therapy events include cardiac arrhythmia such as cardiac fibrillation, respiration rate or level, posture change, activity level, urinary voiding event, fecal voiding event, glucose level, seizure, tremor, gastric activity, sexual activity or any other event that may be used as the basis for adjustment of therapy. The therapy may include any of a variety of therapies such as electrical cardiac stimulation, electrical neurostimulation, electrical muscle stimulation, drug delivery or the like. Electrical neurostimulation therapies may include spinal cord stimulation, deep brain stimulation, gastric stimulation, peripheral nerve stimulation, pelvic floor stimulation and like, which may be provide as therapy for various symptoms or conditions such as chronic pain, tremor, Parkinson's disease, epilepsy, urinary or fecal incontinence, sexual dysfunction, obesity, or gastroparesis.

Upon detection of a therapy event, therapy delivery device 51 of IMD 10 may generate a test command to cause sensing device 2 to perform a self-test (122) to verify that it is functional. Again, sensing device 2 may perform a self-test that activates sensor 4 or a self-test that does not activate sensor 4. Sensing device 2 then determines its operational state (124) by examining the output generated in response to the self-test. The process for determining the operational state of sensor 2 in response to an activating self-test and a non-activating self-test have been described in detail with respect to FIGS. 7 and 8, respectively.

When sensing device 2 is determined to be functional (“YES” branch of decision block 126), sensing device 2 transmits the output to therapy delivery device 51 (134). The output that is transmitted is the output generated by sensing device 2 in accordance with a physiological parameter. Therapy delivery device 51 may then make an operational adjustment, such as adjustment of therapy to a patient, based on the sensor output (136). In particular, therapy delivery device 51 may use the output of sensing device 2 as the primary trigger for an event that drives an operational adjustment in IMD 10, or as a secondary trigger to validate an event triggered by a different sensor. In either case, the operational status of sensing device 2 determines whether therapy delivery device 51 relies on the output of sensing device 2 or not.

When sensing device 2 is determined to be non-functional (“NO” branch of decision block 126), the sensing device may apply a reset feature to attempt to return itself to a functional state. The reset feature begins by determining if device 2 has been reset too many times (decision block 128). If device 2 has been reset too many times, device 2 indicates to an operator or user that device 2 has experienced a failure (step 132). If device 2 has not been reset too many times (“YES” branch of decision block 128), device 2 resets sensor 4 (130) and repeats steps 122, 124, 16, and 128 until device 2 is returned to a functional state or has been reset too many times (128).

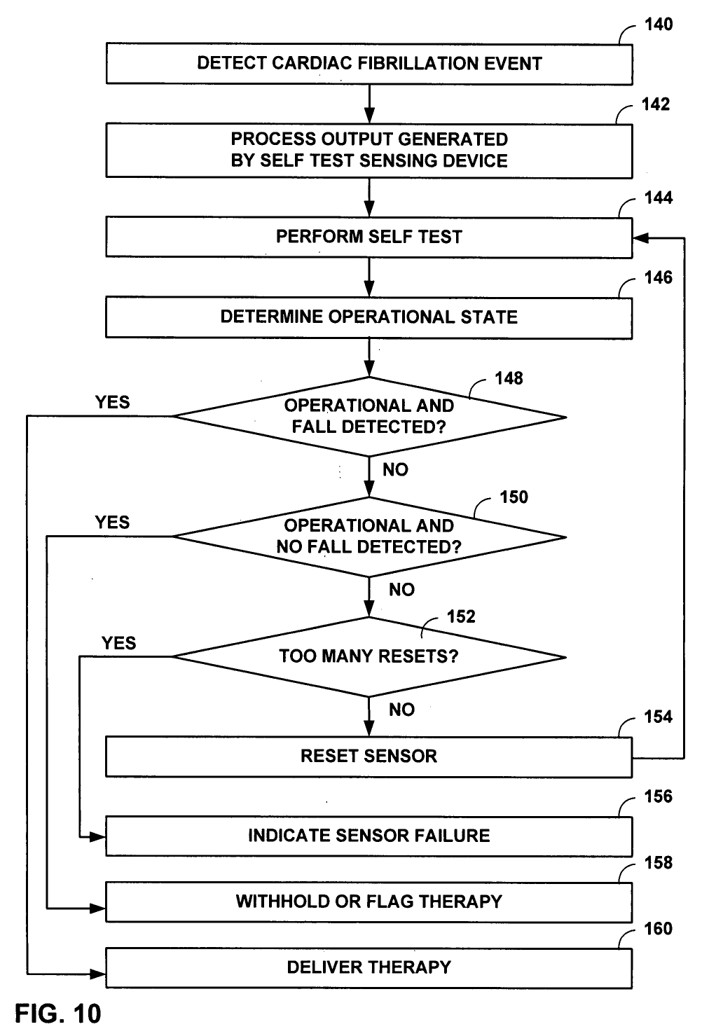

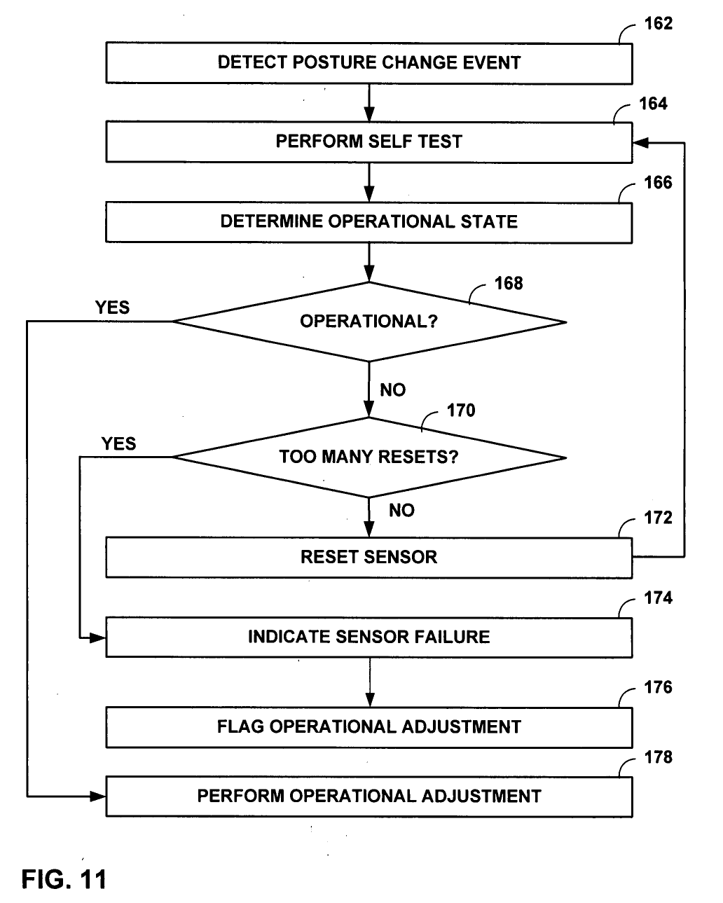

FIG. 10 is a flow diagram illustrating an example technique for using sensing device 2 to validate delivery of therapy in a cardiac rhythm management (CRM) application. In this example, the therapy delivery device 51 may be an implantable cardioverter/defibrillator that includes one or more pressure sensors, ECG sense electrodes, or other sensors for detecting an arrhythmia or cardiac fibrillation. In addition, sensor 4 of sensing device 2 may be a single or multiple axis accelerometer that generates an output signal for use by therapy delivery device 51. The output signal provides information relating to posture, motion and/or activity of the patient. The output of sensing device 2 may be used to control rate-responsive pacing and/or record activity or events such as falls. In addition, the output of sensing device 2 may be used to validate therapy events triggered by other sensing devices.

When a cardiac arrhythmia such as fibrillation is detected, for example, therapy delivery device 51 may examine the output of sensing device 2 to determine if the patient's posture and/or activity is consistent with that of a person experiencing a cardiac event that requires therapy. In other embodiments, sensing device 2 may be a pressure sensor, flow sensor, heart sounds sensor, respiratory sensor or other sensor providing information useful in validating the existence of a cardiac event. In each case, a senor self-test feature may be desirable to ensure that the output of sensing device 2 is accurate, particularly when it is used to support a life-sustaining therapy application, such as cardiac pacing or cardioversion/defibrillation.

Initially, in the example of FIG. 10, therapy delivery device 51 detects a cardiac event (140) in the patient. The cardiac event may be cardiac fibrillation, as detected by sense electrodes and signal analysis electronics within the cardioverter/defibrillator. At the same time, sensing device 2 monitors the posture, motion and/or activity of the patient. Before delivering a defibrillation shock, which may be painful, therapy delivery device 51 may first validate the event with secondary sensors, such as sensing device 2. For example, therapy delivery device 51 may process the output generated by sensing device 2 (142), which may be a self-test accelerometer. In FIG. 9, therapy delivery device 51 process the output of sensing device 2 to determine if the patient experienced a fall coincident with the detection of the cardiac fibrillation, or to determine the posture of patient. Indication of a fall, a sudden change in posture, or a reclined posture may correlate well with cardiac fibrillation, whereas indication of absence of a fall, continued motion or a standing posture may not correlate well with cardiac fibrillation.

Before or after therapy delivery device 51 has processed the output of sensing device 2, sensing device 2 may perform a self-test (144) to determine whether the sensing device is operable, and therefore whether the output is reliable. Sensing device 2 may perform an activating self-test or a non-activating self-test as previously described. Sensor circuitry 6 may determine the operational state (146) of device 2 based on the output generated during the self-test. If the self test indicates that sensing device 2 is operational and the output of sensing device 2 indicates that the patient experienced a sudden fall (“YES” branch of decision block 148), the cardiac fibrillation is validated by sensing device 2. In this case, therapy delivery device 51 may proceed to adjust therapy, e.g., by delivering a cardiac defibrillation shock (160) to the patient.

If the self test indicates that device 2 is operational, but the output of self test sensor 2 does not indicate that the patient experienced a fall (“YES” branch of decision block 150), the patient is less likely to have experienced a cardiac event that requires corrective therapy. In this case, therapy delivery device 51 may either withhold therapy or flag therapy (158). Withholding therapy would be ordinarily be very aggressive, given that fibrillation can lead to imminent death. However, therapy delivery device 51 could divide detected fibrillation waveforms into different categories of shockable rhythms, and rank the categories in terms of reliability as an indicator of fibrillation. In this case, output of sensing device 2 could be used as the basis to withhold therapy for some less reliable waveforms, but give way to therapy for more highly ranked waveforms.

For typical fibrillation waveforms, flagging therapy may be more reasonable than withholding therapy. Flagging therapy may involve temporarily withholding therapy pending further analysis of the cardiac waveform or further analysis of inputs from other sensors, combined with recording of the event in memory for analysis by a medical care-giver. If detection of the cardiac fibrillation persists, therapy delivery device 51 may proceed to delivery a defibrillation shock. Hence, therapy delivery device 51 may deliver therapy even when self-test sensing device 2 is operable and does not validate the detected fibrillation, yet record the discrepancy so that a care-giver may evaluate whether therapy delivery device 51 is suffering from false detection of fibrillation. In this case, the output of sensing device 2 may be used to troubleshoot false detections so that delivery of unnecessary defibrillation shocks can be avoided.

In the case that the self test indicates that sensing device 2 is not operable, the process follows the “NO” branch of decision block 148 and 150, and device 2 is reset (154) if it has not been reset too many times (“NO” branch of decision block 152). The process then repeats steps 144, 146, 148, 150, and 152. However, if sensing device 2 becomes operational after resetting, the steps for determining if therapy should be delivered to the patient are repeated using new output from sensing device 2. If sensing device 2 remains inoperable after being reset the maximum number of times (“YES” branch of decision block 152), sensing device 2 indicates sensor failure (step 156).

FIG. 11 is a flow diagram illustrating an exemplary technique for using a self-test sensing device 2 to trigger or validate a posture change as a medical event. In the example of FIG. 11, sensing device 2 generates a sensor output signal that is received by therapy delivery device 51. Therapy delivery device 51 detects a posture change event based on the sensor output signal (162). The posture change may indicate that the patient has assumed a standing, sitting or reclined position, or indicate that the patient is walking or engaging in activity. In response, therapy delivery device 51 may be configured to make an operational adjustment such as a therapy or diagnostic adjustment.

For example, the operational adjustment may be initiation of delivery of neurostimulation according to a neurostimulation program, an adjustment of one or more neurostimulation parameters associated with a current neurostimulation program, termination of neurostimulation, or selection of a different neurostimulation program. Therapy delivery device 51 may be configured to deliver neurostimulation programs for different postures or activity levels. For example, a patient may receive neurostimulation according to a sitting program, a standing program, a sleeping program, an exercise program, or the like. Each program may specify a different set of neurostimulation parameters, such as neurostimulation pulse amplitude, pulse rate, pulse width, and electrode combination, that are suitable for a given posture.

Transitioning from one program to another may be dependent on posture or activity indications provided by sensing device 2. Accordingly, it is important to ensure that sensing device 2 is operational, and that such output is reliable. To that end, when sensing device 2 triggers a posture change event, motion event, or the like, therapy delivery device 51 may generate a test command that causes sensing device to perform a self-test (164), which may be an activating or non-activating self-test, or both. Sensing device 2 or therapy delivery device 51 determines that operational state of the sensing device (166) based on the result of the self-test. If the sensing device 2 is operational (168), then therapy delivery device 51 performs the operational adjustment dictated by the posture change event (178), e.g., a change from one neurostimulation program to another, an increase or decrease in pulse amplitude, rate or width, and/or a change in the selected electrodes used to deliver neurostimulation.

If the self-test indicates that sensing device 2 is not operational (168) and sensing device 2 has not attempted too many resets (170), then the sensing device may attempt to reset the sensor 4 (172). If resetting is successful, the self-test will indicate that sensing device 2 is operational (166, 168). If too many resets have been attempted (170), then sensing device 2 indicates sensor failure (174). Optionally, therapy delivery device 51 may proceed with the operational adjustment indicated by the posture change event but flag the adjustment for further analysis of additional sensor output signals and/or later review to determine whether the posture change detection was defective.

In the examples of FIGS. 10 and 11, an event associated with an operational adjustment of an implantable medical device is detected. In FIG. 10, the event is cardiac fibrillation detected by another sensing device. In FIG. 11, the event is a posture change or movement sensed by the self-test sensing device 2. In each case, therapy delivery device 51 obtains a sensor signal indicative of a physiological condition from sensing device 2. In response to the detected event, e.g., cardiac fibrillation or posture change, therapy delivery device 51 requests that sensing device 2 perform a self-test, and then determines whether to make an operational adjustment at least in part based on the sensor signal generated by sensing device 2 and a result of the self-test.

For example, therapy delivery device 51 may proceed with the operational adjustment if the sensor signal is consistent with the event and the self-test indicates that the sensing device is operable. If sensing device 2 is operable and generated an output signal consistent with fibrillation, like falling down, therapy delivery device 51 may proceed to deliver a defibrillation shock. If sensing device 2 indicates a posture change, and is self-tested and found to be operable, therapy delivery device 51 likewise may proceed to make an operational adjustment such as a neurostimulation program change. If the sensor signal generated by sensing device 2 is not consistent with a detected event, however, and the self-test indicates that the sensing device is operable, therapy delivery device 51 may withhold or flag the operational adjustment.

As a further refinement, the process for operational adjustment in an IMD 10 may rely on several inputs, including sensor inputs and other operational inputs, to determine whether to make an operational adjustment. If the sensing device 2 produces an output signal that is consistent with or indicates the event associated with an operational adjustment, and the self-test indicates that the sensing device is operable, the output of the sensing device may be given more weight in the operational adjustment process. Alternatively, if the sensing device 2 is found to be inoperable, the output of the sensing device may be given reduced or no weight in the operational adjustment process. Hence, the output of the sensing device 2 may be weighted for purposes of operational adjustment decision-making according to the result of the self-test procedure.

The invention, including self test sensing device 2 and associated devices, systems, methods and IMDs, may be useful in a variety of applications. For example, the invention may be applied to therapies for a variety of symptoms or conditions such as cardiac arrhythmia, cardiac fibrillation, chronic pain, tremor, Parkinson's disease, epilepsy, urinary or fecal incontinence, sexual dysfunction, obesity, or gastroparesis, and may apply to electrical stimulation or drug delivery to a variety of tissue sites, such as the heart, the brain, the spinal cord, pelvic nerves, peripheral nerves, or the gastrointestinal tract of a patient.

Hence, IMD 10 may be a cardioverter/defibrillator, spinal cord stimulator, pelvic nerve stimulator, deep brain stimulator, gastrointestinal stimulator, peripheral nerve stimulator, or muscle stimulator and stimulation may be used in different therapeutic applications, such as cardiac stimulation, deep brain stimulation (DBS), spinal cord stimulation (SCS), pelvic stimulation for pelvic pain, incontinence, or sexual dysfunction, gastric stimulation for gastroparesis, obesity or other disorders, or peripheral nerve stimulation for pain management. Stimulation also may be used for muscle stimulation, e.g., functional electrical stimulation (FES) to promote muscle movement or prevent atrophy.

Self-testing of a sensor device, such as an accelerometer, may be useful for any of such applications. As one example, a fall or posture indicated by an accelerometer may be useful in validating detection of cardiac fibrillation and supporting delivery of defibrillation shocks, or validating detection of an epileptic seizure, and supporting deep brain stimulation to terminate the seizure. As another example, verification of posture may be important in determining whether to automatically adjust stimulation, such as spinal cord stimulation, gastric stimulation, or pelvic stimulation, e.g., when a person is lying down or sleeping, in contrast to working or exercising.

The invention is related to the use of a sensor self-test in conjunction with operation of an IMD. A self-test diagnostic is used to validate that the sensor is still functional while in a patient. The verification of functionality is important to ensure that operational adjustment algorithms that are running in the IMD are using valid information for decision making and actuation. This is particularly important for life sustaining applications, such as defibrillation. The mechanism of self-test may include activation of the sensor itself. Examples of this include electrostatic self-test of MEMS accelerometers and pressure sensors, and potentially small voltages superimposed on EEG or ECG electrodes. The self-test can be run as a periodic scheduled maintenance routine, and/or as a verification during a key decision making algorithm step, e.g., in determining whether a patient fell down so that an IMD should start a loop recorder or provide a therapy, such as a defibrillation shock. The use of self-test can also help streamline manufacturing.

Various embodiments of the invention have been described. These and other embodiments are within the scope of the following claims.

1. An implantable medical device comprising:
a sensing device that generates a sensor signal indicative of a physiological condition; and self-test circuitry that performs a self-test of the sensing device in response to an event associated with an operational adjustment of the implantable medical device.

2. The device of claim 1, wherein the self-test circuitry performs the self-test in response to an event indicated by the sensor signal.

3. The device of claim 1, wherein the self-test circuitry performs the self-test in response to an event indicated by another sensing device.

4. The device of claim 1, further comprising a therapy delivery device that delivers a therapy to a patient, wherein the operational adjustment includes an adjustment of the therapy in response to the event.

5. The device of claim 4, wherein the adjustment of the therapy includes initiation of the therapy, termination of the therapy, or adjustment of one or more parameters associated with the therapy.

6. The device of claim 4, wherein the therapy delivery device includes one of a cardiac stimulation device, a neurostimulation device and a drug delivery device.

7. The device of claim 4, wherein the event includes one of cardiac arrhythmia, patient posture change, patient motion, patient activity level, or patient seizure.

8. The device of claim 4, wherein the self-test circuitry performs the self-test in response to a command from the therapy delivery device.

9. The device of claim 1, further comprising a diagnostic device that performs a diagnostic operation, wherein the operational adjustment includes adjustment of the diagnostic operation in response to the event.

10. The device of claim 9, wherein the diagnostic device records information based on the sensor signal when the self-test circuitry indicates operability of at least one of the sensor and the sensor circuitry.

11. The device of claim 1, wherein the sensing device comprises:
a sensor that generates a signal indicative of a physiological condition; and sensor circuitry that processes the signal to produce the sensor signal, and wherein the self-test circuitry activates the sensor to perform the self-test.

12. The device of claim 11, wherein the sensor is a capacitive-based sensor, the self-test circuitry applying a test signal to the sensor to cause one or more capacitive plates of the sensor to deflect, wherein self-test circuitry detects operability of the sensing circuitry based on the signal generated by the sensor in response to the test signal.

13. The device of claim 1, wherein the sensing device comprises:
a sensor that generates a signal indicative of a physiological condition; and sensor circuitry that processes the signal to produce the sensor signal, and wherein the self-test circuitry does not activate the sensor to perform the self-test.

14. The device of claim 13, wherein the self-test circuitry applies a test signal to the sensor circuitry to perform the self-test.

15. The device of claim 14, wherein the self-test circuitry detects operability of the sensing circuitry based on an output signal generated by the sensor circuitry in response to the test signal.

16. The device of claim 1, wherein the sensor includes an accelerometer.

17. A method comprising:
obtaining a sensor signal indicative of a physiological condition from a sensing device; and
performs a self-test of the sensing device in response to an event associated with an operational adjustment of an implantable medical device.

18. The method of claim 17, further comprising performing the self-test in response to an event indicated by the sensor signal.

19. The method of claim 17, further comprising performing the self-test in response to an event indicated by another sensor.

20. The method of claim 17, further comprising delivering a therapy to a patient via the implantable medical device, wherein the operational adjustment includes an adjustment of the therapy in response to the event.

21. The method of claim 20, wherein the adjustment of the therapy includes initiation of the therapy, termination of the therapy, or adjustment of one or more parameters associated with the therapy.

22. The method of claim 20, wherein the therapy includes one of cardiac stimulation device, neurostimulation and drug delivery.

23. The method of claim 20, wherein the event includes one of cardiac arrhythmia, patient posture change, patient motion, patient activity level, or patient seizure.

24. The method of claim 20, further comprising performing the self-test in response to a command from a therapy delivery device.

25. The method of claim 17, further comprising performing a diagnostic operation, wherein the operational adjustment includes adjustment of the diagnostic operation in response to the event.

26. The method of claim 25, wherein the diagnostic operation includes recording information based on the sensor signal when the self-test indicates operability of at least one of the sensor and the sensor circuitry.

27. The method of claim 17, wherein the sensing device comprises:
a sensor that generates a signal indicative of a physiological condition; and sensor circuitry that processes the signal to produce the sensor signal, the method further comprising activating the sensor to perform the self-test.

28. The method of claim 27, wherein the sensor is a capacitive-based sensor, the method further comprising applying a test signal to the sensor to cause one or more capacitive plates of the sensor to deflect, and detecting operability of the sensing circuitry based on the signal generated by the sensor in response to the test signal.

29. The method of claim 17, wherein performing the self-test comprises not activating the sensor to perform the self-test.

30. The method of claim 29, wherein the sensing device comprises:
a sensor that generates a signal indicative of a physiological condition; and sensor circuitry that processes the signal to produce the sensor signal, the method further comprising applying a test signal to the sensor circuitry to perform the self-test.

31. The method of claim 30, wherein performing the self-test includes detecting operability of the sensing circuitry based on an output signal generated by the sensor circuitry in response to the test signal.

32. The method of claim 17, wherein the sensor includes an accelerometer.

33. An implantable medical device comprising:
means for obtaining a sensor signal indicative of a physiological condition from a sensing device; and
means for performing a self-test of the sensing device in response to an event associated with an operational adjustment of an implantable medical device.

34. A method comprising:
detecting an event associated with an operational adjustment of an implantable medical device;
obtaining a sensor signal indicative of a physiological condition from a sensing device;
performing a self-test of the sensing device in response to the event; and
determining whether to make the operational adjustment based on the sensor signal and a result of the self-test.

35. The method of claim 34, further comprising proceeding with the operational adjustment if the sensor signal is consistent with the event and the self-test indicates that the sensing device is operable.

36. The method of claim 34, further comprising proceeding with the operational adjustment if the sensor signal indicates the event and the self-test indicates that the sensing device is operable.

37. The method of claim 34, further comprising withholding the operational adjustment if the sensor signal is not consistent with the event and the self-test indicates that the sensing device is operable.

38. The method of claim 34, further comprising proceeding with the operational adjustment if the event is indicated by another sensing device and the self-test indicates that the sensing device is operable.

39. The method of claim 34, wherein the operational adjustment includes an adjustment to therapy delivered by the implantable medical device.

40. An implantable medical device comprising:
a sensing device that generates a sensor signal indicative of a physiological condition; and
a therapy delivery device that detects an event associated with an operational adjustment of an implantable medical device,
wherein the sensing device performs a self-test of the sensing device in response to the event, and
the therapy delivery device determines whether to make the operational adjustment based on the sensor signal and a result of the self-test.

41. The device of claim 40, wherein the therapy delivery device proceeds with the operational adjustment if the sensor signal is consistent with the event and the self-test indicates that the sensing device is operable.

42. The device of claim 40, wherein the therapy delivery device proceeds with the operational adjustment if the sensor signal indicates the event and the self-test indicates that the sensing device is operable.

43. The device of claim 40, wherein the therapy delivery device withholds the operational adjustment if the sensor signal is not consistent with the event and the self-test indicates that the sensing device is operable.

44. The device of claim 40, wherein the therapy delivery device proceeds with the operational adjustment if the event is indicated by another sensing device and the self-test indicates that the sensing device is operable.

45. The device of claim 40, wherein the operational adjustment includes an adjustment to therapy delivered by the implantable medical device.

46. An implantable medical device comprising:
means for detecting an event associated with an operational adjustment of an implantable medical device;
means for obtaining a sensor signal indicative of a physiological condition from a sensing device;
means for performing a self-test of the sensing device in response to the event; and
means for determining whether to make the operational adjustment based on the sensor signal and a result of the self-test.
